(19) United States
(12) Patent Application Publication
(10) Pub. No.: US 2008/0064635 A1
(43) Pub. Date: Mar. 13, 2008

(54) Infant Formulas for Early Brain Development

(76) Inventors: Ricardo Rueda, Granada (ES); Alejandro Barranco, Las Gabias (ES); Maria Ramirez, Granada (ES); Enrique Vazquez, Ogijares (ES); Eduardo Valverde, Cajar (ES); Pedro Prieto, Mexico City (ES); Margaret H. Dohnalek, Gurnee, IL (US)

Correspondence Address: ROSS PRODUCTS DIVISION OF ABBOTT LABORATORIES;DEPARTMENT 108140-DS/1, 625 CLEVELAND AVENUE, COLUMBUS, OH, 43215-1724, US

(21) Appl. No.: 11/771,417

(22) Filed: Jun. 29, 2007

Related U.S. Application Data
(63) Continuation-in-part of application No. 11/479,621, filed on Jun. 30, 2006.

(51) Int. Cl. A61K 38/16; A23D 7/005; A61P 43/00

(52) U.S. Cl. 514/12; 426/601

(57) ABSTRACT

Disclosed are infant formulas comprising at least about 6.5 g/L, on an as-fed basis, of an enriched whey protein concentrate, at least about 0.13% docosahexaenoic acid by weight of total fatty acids, and at least about 0.25% arachidonic acid by weight of total fatty acids. The formulas also typically include at least about 5 mg/L of gangliosides, at least about 150 mg/L of phospholipids, and at least about 70 mg/L of total sialic acid with at least about 2.5% as lipid-bound sialic acid, all of which are provided in whole or in part from the enriched whey protein concentrate. Also disclosed are methods of accelerating brain development, neural migration, and cognitive development in an infant by administering the infant formulas during the first 2-4 months of life, preferably as a sole source of nutrition.

### CROSS-REFERENCE TO RELATED APPLICATION

This application is a continuation-in-part of U.S. patent application Ser. No. 11/479,621, filed on Jun. 30, 2006.

### TECHNICAL FIELD

The present invention relates to infant formulas comprising select combinations of enriched whey protein concentrate, docosahexaenoic acid and arachidonic acid to better assimilate the natural composition of human milk and to accelerate early brain development in infants

### BACKGROUND OF THE INVENTION

Commercial infant formulas are commonly used today to provide supplemental or sole source nutrition early in life. These formulas comprise a range of nutrients to meet the nutritional needs of the growing infant, and typically include fat, carbohydrate, protein, vitamins, minerals, and other nutrients helpful for optimal infant growth and development.

Commercial infant formulas are designed to assimilate, as closely as possible, the composition and function of human milk. In the United States, the Federal Food, Drug, and Cosmetic Act (FFDCA) defines infant formula as “a food which purports to be or is represented for special dietary use solely as a food for infants by reason of its simulation of human milk or its suitability as a complete or partial substitute for human milk.” (FFDCA 201(z)).

Commercial infant formulas, under FFDCA rules, are defined by basic nutrients that must be formulated into non-exempt infant formulas in the U.S. These nutrients include, per 100 kcal of formula: protein (1.8-4.5 g at least nutritionally equivalent to casein), fat (3.3-6.0g), linoleic (at least 300 mg), vitamin A as retinol equivalents (75-225 mcg), vitamin D (40-100 IU), vitamin K (at least 4.0 mcg), vitamin E (at least 0.7 IU/g linoleic acid), ascorbic acid (at least 8.0 mg), thiamine (at least 40 mcg), riboflavin (at least 60 mcg), pyridoxine (at least 35.0 mcg with 15 mog/g of protein in formula), vitamin B12 (at least 0.15 mcg), niacin (at least 250 mcg), folic acid (at least 4.0 mcg), pantothenic acid (at least 300.0 mcg), biotin (at least 1.5 mcg), choline (at least 7.0 mg), inositol (at least 4.0 mg), calcium (at least 50.0 mg), phosphorous (at least 25.0 mg with calcium to phosphorous ratio of 1.1-2.0), magnesium (at least 6.0 mg), iron (at least 0.15 mg), iodine (at least 5.0 mcg), zinc (at least 0.5 mg), copper (at least 60.0 mcg), manganese (at least 5.0 mcg), sodium (20.0-60.0 mg), potassium (80.0-200.0 mg), and chloride (55.0-150.0 mg).

Notwithstanding tight regulatory controls, commercial infant formulas are still not identical, in either composition or function, to human milk. Almost 200 different compounds have been identified in human milk, over 100 of which are still not typically found in significant amounts, or at all, in commercial formulas. Such compounds include various immunoglobulins, enzymes, hormones, certain proteins, lactoferrin, gangliosides, phospholipids (sphingomyelin, phosphatidyl ethanolamine, phosphatidyl choline, phosphatidyl serine, phosphatidyl inositol), and so forth. Many of these materials are unique to human milk or are otherwise present in only minor concentrations in cow's milk or other protein sources used in preparing a commercial infant formula.

There is a continuing need, therefore, for new infant formulas that contain even more of the natural ingredients found in human milk, to thus potentially provide more of the nutritional benefits currently enjoyed by the breastfed infant.

The present invention is directed to infant formulas with select concentrations and types of those compounds inherently found in human milk, including docosahexaenoic acid, arachidonic acid, phospholipids, gangliosides, and sialic acid. By virtue of these selected ingredients and their corresponding concentrations in the infant formulas, the nutrient profiles of the infant formulas described herein are more similar to human milk than are conventional infant formulas.

It was discovered, however, that not only do these formulas better assimilate the natural ingredients found in human milk, but they may also accelerate neuroblast migration during the first 3-4 months of life, thus providing an infant formula that helps accelerate brain and cognitive development in infants. Interestingly, the effect on neuroblast migration was only noted during the early infancy phase (see animal study described herein) thus emphasizing the importance of the selected use of these formulas during this early infancy phase.

### SUMMARY OF THE INVENTION

A first embodiment of the present invention is directed to infant formulas comprising at least about 6.5 g/L, on an as-fed basis, of enriched whey protein concentrate, and at least about 0.13% docosahexaenoic acid by weight of total fatty acids, and at least about 0.25% arachidonic acid by weight of total fatty acids. The formulas may also include on an as-fed basis at least about 5 mg/L of gangliosides, at least about 150 mg/L of phospholipids, and at least about 70 mg/L of total sialic acid with at least about 2.5% as lipid-bound sialic acid, all of which may be provided, in whole or in part, from the enriched whey protein concentrate

A second embodiment of the present invention is directed to a method of accelerating neuroblast migration during the first 2-4 months of life, said method comprising the oral administration of an infant formula comprising at least about 6.5 g/L, on an as-fed basis, of enriched whey protein concentrate, at least about 0.13% docosahexaenoic acid by weight of total fatty acids, and at least about 0.25% arachidonic acid by weight of total fatty acids. The formulas may also include at least about 5 mg/L of gangliosides, at least about 150 mg/L of phospholipids, and at least about 70 mg/L of total sialic acid with at least about 2.5% as lipid-bound sialic acid, all of which may be provided, in whole or in part, from the enriched whey protein concentrate.

A third embodiment of the present invention is directed to a method of accelerating cognitive development in an infant, especially during the first 24 months of life, said method comprising the oral administration of an infant formulas comprising at least about 6.5 g/L, on an as-fed basis, of enriched whey protein concentrate, at least about 0.13% docosahexaenoic acid by weight of total fatty acids, and at least about 0.25% arachidonic acid by weight of total fatty acids. The formulas may also include at least about 5 mg/L of gangliosides, at least about 150 mg/L of phospholipids, and at least about 70 mg/L of total sialic acid with at least about 2.5% as lipid-bound sialic acid, all of which may be provided, in whole or in part, from the enriched whey protein concentrate.

It was discovered that, not only do these formulas better assimilate the natural ingredients found in human milk; they also accelerate neuroblast migration during the early phase of infancy, thus providing an infant formula that helps accelerate brain and cognitive development in infants. Interestingly, the effect on neuroblast migration was only noted during the early infancy phase (see animal study described herein) thus emphasizing the importance of the selected use of these formulas during the first 2-4 months of life.

It was also discovered that the effect on neuroblast migration occurred only when the enriched whey protein concentrate was used in combination with higher levels of docosahexaenoic acid and arachidonic acids. Identical compositions, but with lower concentrations of docosahexaenoic acid and arachidonic acids, did not significantly affect neuroblast migration in the selected animal model.

It was also discovered that the effect on neuroblast migration occurred only when the infant formulas comprise a level of enriched whey protein concentrate that exceeds a minimum threshold amount as defined herein.

### BRIEF DESCRIPTION OF THE DRAWINGS

FIG. 1.1 shows a pig brain segment for histological measurements in the animal study described herein (Experiment 1).

FIG. 1.2 is a magnified section of the FIG. 1.1 pig brain section, which shows subependymal area stained with hematoxilin:eosin; darker stained dots are nuclei; neuroblasts migrate from the subependymal area to the white matter (Experiment 1).

FIG. 1.3 shows Areas 1, 2 and 3 from the FIG. 12 magnified pig brain section for nucleus counts; Area 1 is the subrallosal fasciculus, neuroblast migration and proliferation area; Area 2 is the migration area avoiding neuroblast aggregates; and Area 3 is the white matter next to the subcallosal fasciculus (Experiment 1).

FIG. 2 includes three graphs corresponding to the nuclei count for Area 1, Area 2, and Area 3 of the subcallosal fasciculus in piglets fed with the different diets (A, B, C) during the period of study described herein. Data are Mean±SD. a: significantly different from initial time at p<005; b: significantly different from 8-9 d at p<0.05; *: significantly different from diet A at p<0.05 (Experiment 1).

FIG. 3.1 includes a graph corresponding to the number of nuclei stained with H&E in the subcallosal fasciculus of piglets fed the different diets (A, B, C) or sow's milk (Experiment 11).

FIG. 3.2 includes a graph corresponding to the number of nuclei stained with H&E in the white matter adjacent to the subcallosal fasciculus of piglets fed the different diets (A, B, C) or sow's milk (Experiment II).

FIG. 3.3 includes a graph corresponding to the number of BrdU positive cells in the subeallosal fasciculus of piglets fed the different diets (A, B, C) or sow's milk (Experiment II).

FIG. 4.1 includes a graph corresponding to the number of BrdU positive cells in the white matter adjacent to the subeallosal fasciculus of piglets fed the different diets (A, B, C) or sow's milk (Experiment II).

FIG. 4.2 includes a graph corresponding to the number of Ki67 positive cells in the subcallosal fasciculus of piglets fed the different diets (A, B, C) or sow's milk (Experiment II).

FIG. 4.3 includes a graph corresponding to the number of Ki67 positive cells in the white matter adjacent to the subcallosal fasciculus of piglets fed the different diets (A, B, C) or sows milk (Experiment II).

### DETAILED DESCRIPTION OF THE INVENTION

The compositions of the present invention comprise select combinations of enriched whey protein concentrate, docosahexaenoic acid, and arachidonic acids, each of which is described in detail hereinafter.

The term “infant” as used herein refers to individuals not more than about one year of age, and includes infants from 0 to about 4 months of age, infants from about 4 to about 8 months of age, infants from about 8 to about 12 months of age, low birth weight infants at less than 2,500 grams at birth, and preterm infants born at less than about 37 weeks gestational age, typically from about 26 weeks to about 34 weeks gestational age.

The term “as fed” as used herein, unless otherwise specified, refers to liquid formulas suitable for direct oral administration to an infant, wherein the formulas are ready-to-feed liquids, reconstituted powders, or diluted concentrates.

The term “infant formula” as used herein, unless otherwise specified, refers to formulations comprising fat, protein, carbohydrates, vitamins, and minerals, and that are suitable for oral administration to infants as supplemental, primary, or sole sources of nutrition, non limiting examples of which include reconstitutable powders, dilutable concentrates, and ready-to-feed liquids.

All ingredient ranges as used herein, unless otherwise specified, to characterize the infant formulas of the present invention are by weight of infant formula on an as-fed basis.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

The infant formulas of the present invention may also be substantially free of any optional or selected essential ingredient or feature described herein, provided that the remaining formula still contains all of the required ingredients or features as described herein. In this context, and unless otherwise specified, the term “substantially free” means that the selected composition contains less than a functional amount of the optional ingredient, typically less than 0.1% by weight, and also including zero percent by weight of such optional or selected essential ingredient.

All references to singular characteristics or limitations of the present invention shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present invention, including components thereof, can comprise, consist of, or consist essentially of the essential elements and limitations of the invention described herein, as well as any additional or optional ingredients, components, or limitations described herein or otherwise useful in nutritional formula applications.

### Enriched Whey Protein Concentrate

The infant formulas of the present invention comprise selected levels of enriched whey protein concentrates as a source of gangliosides, phospholipids, and sialic acid in the infant formula. All or part of such gangliosides, phospholipids, and sialic acid in the formula may be provided by the enriched whey protein concentrate.

The level of enriched whey protein concentrate in the infant formula must exceed about 6.5 g/L of formula, on an as-fed basis. Such concentrations may also range from about 6.5 to about 10.9 g/L, including from about 6.6 to about 8.5 g/L, and also including from about 6.7 to about 7.3 g/L, of the formula, on an as fed basis.

The enriched whey protein concentrates for use in the infant formulas of the present invention are those having a high concentration of milk fat globule membrane materials. Milk fat globule membrane materials are the membrane and membrane-associated materials that surround the triacylglycerol-rich milk fat globules in bovine or other mammalian milk. Many of the compounds identified in the milk fat globule membrane materials are present in much higher concentrations in human milk than in commercial infant formulas. By adding whey protein concentrates enriched in such materials to an infant formula, the resulting formula is more similar in composition to human milk, especially with respect to human milk concentrations of gangliosides, phospholipids, and sialic acid.

The term “enriched whey protein concentrate” as used herein, unless otherwise specified, refers generally to any whey protein concentrate having at least about 3%, more typically at least about 5%, by weight of phospholipids, of which at least about 20% by weight of sphingomyelin; at least about 0.5%, typically at least about 1.2% by weight of a sialic acid; and at least about 0.05%, typically at least about 0.1%, by weight of gangliosides. At least about 2.5% by weight of the sialic acid from the concentrate is lipid-bound.

Suitable sources of enriched whey protein concentrate for use herein include any whey protein concentrate having the above-described levels of enriched ingredients, non-limiting examples of which include LACPRODAN® MFGM-10, Whey Protein Concentrate, available from Aria Food Ingredients, Denmark, which contains 6.5% phospholipids, 0.2% gangliosides, 1.80% sialic acid (at least 2.5% lipid-bound sialic acid by weight of total fatty acids), and 1.5% lactoferrin, by weight of the concentrate.

The enriched whey protein concentrate preferably provides from about 10% to 100%, including from about 50% to about 100%, also including from about 50% to about 90%, and also including from about 60% to about 85%, of the total phospholipid, ganglioside, and sialic acid in the infant formula. Although the latter compounds can be added individually, as isolated compounds from mammalian milk or other suitable sources, it is preferred that most if not all of such compounds be provided by the enriched whey protein concentrate.

### Sialic Acid

The infant formulas of the present invention may comprise sialic acid at a concentration, on an as fed basis, of at least 70 mg/L, including from about 90 mg/L to about 4000 mg/l, also including from about 190 mg/liter to about 2000 mg/l, also including from about 300 mg/L to about 900 mg/L, wherein at least 2.5%, including from about 2.6% to about 10%, including from about 2.7% to about 5%, by weight of the sialic acid is lipid-bound. Some or all of the sialic acid may be provided by the enriched whey protein concentrate as described herein.

The lipid-bound sialic acid component of the infant formula is most typically in the form of a ganglioside, which inherently contain lipid-bound sialic acid. The ganglioside component of the present invention, as described hereinafter, may therefore be a primary or sole source of the lipid-bound sialic acid component of the present invention.

The term “sialic acid” as used herein, unless otherwise specified, refers to all conjugated and non-conjugated forms of sialic acid, including sialic acid derivatives. The sialic acid in the infant formula of the present invention may therefore include free sialic acid, protein-bound sialic acid, lipid-bound sialic acid (including gangliosides), carbohydrate-bound sialic acid, and combinations or derivatives thereof. All sialic acid concentrations described herein are based upon the weight percentage of the sialic acid compound or moiety itself, less protein, lipid, carbohydrate, or other conjugates bound to the sialic acid structure.

Sialic acid sources for use in the infant formulas may be added or obtained as separate ingredients. More typically, however, the sialic acid is provided primarily as an inherent ingredient from a whey protein concentrate component, preferably from an enriched whey protein concentrate as described herein. Although less preferred, sialic acid may be obtained from and added as a separate ingredient to the infant formula, in which case the added sialic acid is combined with inherent sialic acid from other ingredients to provide the total sialic acid content in the infant formula.

As an individual compound or moiety, sialic acid is a 9 carbon amino sugar, the structure of which is readily described in the chemical literature. Other generally accepted names for N-acetylneuraminic acid include sialic acid; o-Sialic acid; 5-Acetamido-3,5dideoxy-D-glycero-D-galacto-2-nonulosonic acid; 5-Acetamido-3,5-dideoxy-D-glycero-D-galactonulosonic acid; Aceneuramic acid; N-acetyl-neuraminate; N-Acetylneuraminic acid; NANA; NANA, Neu5Ac; and Neu5Ac.

Suitable sialic acid sources may be either natural or synthetic, and include any of the more than 40 naturally occurring and currently identified sialic acid derivatives, which includes free sialic acid, oligosaccharide conjugates (e.g. sialyloligosaccharides), lipid conjugates (i.e., glycolipids), protein conjugates (i.e., glycoproteins), and combinations thereof.

Sialic acid suitable for use herein includes sialyloligosaccharides commonly found in human milk, whether natural or synthetic, the two most abundant of which are 3′sialyllactose (3′SL, NeuNAcα2-3Galactoseβ1-4Glucose) and 6′sialyllactose (6′SL, NeuNAcα2-6Galactoseβ1-4Glucose). Other suitable sialyloligosaccharides include those that contain one or more sialic acid molecules conjugated to larger human milk or other more complex oligosaccharides.

Other suitable sialic acids for use herein include any corresponding glycolipid that is also suitable for use in an infant formula, including gangliosides such as sialic acid-containing glycolipids comprising a fatty acid, sphingosine, glucose, galactose, N-acetylgalactosamine, N-acetylglucosamine, and N-acetylneuraminic acid molecule. These sialic acid compounds may also include any one or more of the several glycoproteins commonly found in human milk that are known to be sialylated (e.g., κ-casein, α-lactalbumin, lacgtoferrin)

Suitable sources of sialic acid for use herein include isolates, concentrates, or extracts of mammalian milk or milk products, including human and bovine milk. Bovine milk is a preferred source for use herein, including enriched whey protein concentrates as described herein. individual sources of sialic acid suitable for use herein includes Lacprodan CGMP-10 (caseino glyco macropeptide with 4.2% sialic acid), available from Aria Food Ingredients, Denmark; and Biopure glycomacropeptide (with 7-8% sialic acid), available from Davisco Foods International, Eden Prairie, Minn., USA.

Although the infant formulas may comprise glycomacropeptides as a source of sialic acid, the formulas are preferably substantially reduced in glycomacropeptide content. Glycomacropeptide is part of the bovine milk protein casein molecule. Only very small amounts of free glycomacropeptide are found in skim milk, but whey protein concentrate contains higher amounts of free glycomacropeptide. It has been found that glycomacropeptides are not tolerated by infants as well as other sialic acid sources. Thus, infant formulas made with whey protein concentrate have higher free glycomacropeptide content, but also could be less well tolerated by the infant. In this context, the term “substantially reduced” means that the infant formulas preferably contain less than 0.5%, including less than 0.4%, and also including less than 0.35%, and also including zero percent, by weight of the formula as free glycomacropeptide on an as-fed basis. Conventional infant formulas typically contain from 0.6 to 0.8% glycomacropeptide as an inherent ingredient from a typical whey protein concentrate from cheese whey.

### Gangliosides

The infant formulas of the present invention may also comprise enriched concentrations of one or more gangliosides, a group of compounds composed of a glycosphingolipid (ceramide and oligosaccharide) with one or more sialic acids (n-acetylneuraminic acid) linked to the oligosaccharide chain. Some or all of the gangliosides may be provided by the enriched whey protein concentrate as described herein.

Gangliosides are normal components of plasma membranes of mammalian cells and are particularly abundant in neuronal membranes. They are acidic glycosphingolipids comprising a hydrophobic portion, the ceramide, and a hydrophilic portion, an oligosaccharide chain containing one or more molecules of sialic acid. The oligosaccharide moieties of the gangliosides have different chemical structures constituting the reference basis for gangliosides separation and their recognition as individual entities. The ceramide moiety of the most common gangliosides has a heterogeneous fatty acid composition with a prevalence of C18 and C20 derivatives.

Gangliosides are most commonly named using M, D and T designations, which refer to mono-, di- and trisialogangliosides, respectively, and the numbers 1, 2, 3, etc refer to the order of migration of the gangliosides on thin-layer chromatography. For example, the order of migration of monosialogangliosides is GM3>GM2>GM1. To indicate variations within the basic structures, further subscripts are added, e.g. GM1a, GD1b, etc.

The infant formulas of the present invention may comprise at least about 5 mg/L of gangliosides, including from about 7 mg/L to 50 mg/L, also including from about 10 to about 30 mg/L. These ganglioside concentrations are similar to that found in human milk, which typically contains at least about 3 mg/L of gangliosides, more typically from about 3 mg/L to about 30 mg/L of gangliosides. These gangliosides for use in the infant formulas typically comprise one or more, more typically all, of the gangliosides GD3, O-Acetyl-G03 and GM3. These gangliosides generally represent at least about 80%, more typically at least about 90%,by weight of the total gangliosides in the infant formula herein.

Suitable sources of gangliosides for use herein include isolates, concentrates, or extracts of mammalian milk or milk products, including human and bovine milk. Bovine milk is a preferred ganglioside source for use herein, including enriched whey protein concentrates as described herein.

Individual sources of gangliosides suitable for use herein include Ganglioside 500 (>0.5% GM3 and <1.0% GD3) and Ganglioside 600 (>1.2% GD3), available from Fonterra, New Zealand.

Ganglioside concentrations for purposes of defining the infant formulas of the present invention are measured in accordance with the ganglioside method described hereinafter.

### Phosphopids

The infant formulas of the present invention may also comprise enriched concentrations of phospholipids. Such concentrations are higher than that found in conventional infant formulas but similar to that found in human milk. Some or all of the phospholipids may be provided by the enriched whey protein concentrate as described herein.

Phospholipids suitable for use herein include those commonly found in bovine and other mammalian milk. Preferred phospholipids include sphingomyelin, phosphatidyl ethanolamine, phosphatidyl choline, phosphatidyl inositol, phosphatidyl serine, and combinations thereof Most preferred are combinations of all five phospholipids, especially such combinations in which sphingomyelin represents at least 20% by weight of total phospholipids.

Phospholipid concentrations in the infant formulas of the present invention may be at least about 150 mg/L, including from about 200 mg/L to about 600 mg/L, also including from about 250 to about 450 mg/L. Human milk, for comparison, generally contains from about 163 to about 404 mg/L of phospholipids, with sphingomyelin representing about 51% of the total phospholipids.

Suitable sources of phospholipids for use herein include isolates, concentrates, or extracts of mammalian milk or milk products, including human and bovine milk. Bovine milk is a preferred phospholipid source for use herein, including enriched whey protein concentrates as described herein.

Other suitable phospholipid sources include soy, such as soy lecithin. The infant formulas of the present invention, however, are preferably substantially free of phospholids from soy sources. The infant formulas are also preferably substantially free of egg phospholipids. In this context, the term “substantially free” means that the infant formulas contain less than 0.5%, more preferably less than 0.1%, including zero percent, by weight of soy or egg phospholipids.

Individual sources of phospholipids suitable for use herein include milk derived sources such as Phospholipid concentrate 600 (>18.0% Sphingomyelin, >36.0% Phosphatidyl Choline, >9.0% Phosphatidyl Ethanolamine, 4.0% Phosphatidylserine), available from Fonterra, New Zealand.

### Docosahexaenoic and Arachidonic Acids

The infant formulas of the present invention further comprise docosahexaenoic acid and arachidonic acid or sources thereof, wherein the formula must contain at least about 0.13% docosahexaenoic acid and at least about 0.25% arachidonic acid. These two polyunsaturated fatty acids are also found in human milk.

The infant formulas of the present invention must therefore contain arachidonic acid, minimum concentrations of which must be at least about 0.25%, preferably at least about 0.3%, more preferably at least about 0.4%, by weight of total fatty acids in the formula. Arachidonic acid concentrations in the infant formula may range up to about 2.0%, including up to about 1.0%, also including up to about 0.6%, by weight of the total fatty acids in the formula.

The infant formulas of the present invention must likewise contain docosahexaenoic acid, minimum concentrations of which must be at least about 0.13%, preferably at least about 0.14%, more preferably at least about 0.15%, by weight of total fatty acids in the formula. Docosahexaenoic acid concentrations in the infant formula may range up to about 1.0%, including up to about 0.5%, also including up to about 0.25%, by weight of the total fatty acids in the formula.

Non-limiting examples of some suitable sources of arachidonic acid, and/or docosahexaenoic acid include marine oil, egg derived oils, milk fat, fungal oil, algal oil, other single cell oils, and combinations thereof. The compositions are preferably substantially free of egg derived oils, which in this context means less than about 0.05%, including zero percent, by weight of such egg derived oils.

Arachidonic and docosahexaenoic acids may be added to the formula in any form that is suitable for use by an infant, including compounds or materials that can otherwise provide a source of such free fatty acids upon or following administration to the infant, including phospholipids and glyceride esters (mono-, di-, tri-) of polyunsaturated fatty acids. Polyunsaturated fatty acids and sources thereof are described in U.S. Pat. No. 6,080,787 (Carlson, et al.) and U.S. Pat. No. 6,495,599 (Auestad, et al.), which descriptions are incorporated by reference herein. For purposes of defining the present invention, phospholipid sources of arachidonic and docosahexaenoic acid are not included as a phospholipid component as described hereinbefore.

These fatty acids are also described in U.S. Pat. No. 6,495,599 (Auestad et al.), which description is incorporated herein by reference.

### Other Nutrients

The infant formulas of the present invention comprise fat, protein, carbohydrate, vitamins and minerals, all of which are selected in kind and amount to meet the nutrition needs of the targeted infant or defined infant population.

Many different sources and types of carbohydrates, fats, proteins, minerals and vitamins are known and can be used in the base formulas herein, provided that such nutrients are compatible with the added ingredients in the selected formulation and are otherwise suitable for use in an infant formula.

Carbohydrates suitable for use in the formulas herein may be simple or complex, lactose-containing or lactose-free, or combinations thereof, non-limiting examples of which include hydrolyzed, intact, naturally and/or chemically modified cornstarch, maltodextrin, glucose polymers, sucrose, corn syrup, corn syrup solids, rice or potato derived carbohydrate, glucose, fructose, lactose, high fructose corn syrup and indigestible oligosaccharides such as fructooligosaccharides (FOS), galactooligosaccharides (GOS), and combinations thereof.

Proteins suitable for use in the formulas herein include hydrolyzed, partially hydrolyzed, and non-hydrolyzed or intact proteins or protein sources, and can be derived from any known or otherwise suitable source such as milk (e.g., casein, whey, human milk protein), animal (e.g., meat, fish), cereal (e.g., rice, corn), vegetable (e.g., soy), or combinations thereof.

Proteins for use herein may also include, or be entirely or partially replaced by, free amino acids known for or otherwise suitable for use in infant formulas, non-limiting examples of which include alanine, arginine, asparagine, carnitine, aspartic acid, cystine, glutamic acid, glutamine, glycine, histidine, isoleucine, leucine, lysine, methionine, phenylalanine, proline, serine, taurine, threonine, tryptophan, taurine, tyrosine, valine, and combinations thereof. These amino acids are most typically used in their L-forms, although the corresponding D-isomers may also be used when nutritionally equivalent. Racemic or isomeric mixtures may also be used.

Fats suitable for use in the formulas herein include coconut oil, soy oil, corn oil, olive oil, safflower oil, high oleic safflower oil, algal oil, MCT oil (medium chain triglycerides), sunflower oil, high oleic sunflower oil, palm and palm kernel oils, palm olein, canola oil, marine oils, cottonseed oils, and combinations thereof. The infant formulas of the present invention include those embodiments comprising less than about 1%, including less than about 0.2%, including zero percent, by weight of milk fat on an as-fed basis.

Vitamins and similar other ingredients suitable for use in the formulas include vitamin A, vitamin D, vitamin E, vitamin K, thiamine, riboflavin, pyridoxine, vitamin B12, niacin, folic acid, pantothenic acid, biotin, vitamin C, choline, inositol, salts and derivatives thereof, and combinations thereof.

Minerals suitable for use in the base formulas include calcium, phosphorus, magnesium, iron, zinc, manganese, copper, chromium, iodine, sodium, potassium, chloride, and combinations thereof.

The infant nutrition formulas of the present invention preferably comprise nutrients in accordance with the relevant infant formula guidelines for the targeted consumer or user population, an example of which would be the Infant Formula Act, 21 U.S.C. Section 350(a). Preferred carbohydrate, lipid, and protein concentrations for use in the formulas are set forth in the following table.

TABLE 1
Macronutrient Ranges

| | | gm/100 | gm/100 | |
|---|---|---|---|---|
| Nutrient | Range 1 | kcal | gm powder | gm/liter as fed |
| Carbohydrate | Preferred | 8-16 | 30-90 | 54-108 |
| | More preferred | 9-13 | 45-60 | 61-88 |
| Lipid | Preferred | 3-8 | 15-35 | 20-54 |
| | More preferred | 4-6.6 | 25-25 | 27-45 |
| Protein | Preferred | 1-3.5 | 8-17 | 7-24 |
| | More preferred | 1.5-3.4 | 10-17 | 10-23 |

All numerical values are preferably modified by the term “about”

The infant formulas may also include per 100 kcal of formula one or more of the following: vitamin A (from about 250 to about 750 IU), vitamin D (from about 40 to about 100 IU), vitamin K (greater than about 4 μm), vitamin E (at least about 0.3 IU), vitamin C (at least about 8 mg), thiamine (at least about 8 μg), vitamin B12 (at least about 0.15 μg), niacin (at least about 250 μg), folic acid (at least about 4 μg), pantothenic acid (at least about 300 μg), biotin (at least about 1.5 μg), choline (at least about 7 mg), and inositol (at least about 2 mg).

The infant formulas may also include per 100 kcal of formula one or more of the following: calcium (at least about 50 mg), phosphorus (at least about 25 mg), magnesium (at least about 6 mg), iron (at least about 0.15 mg), iodine (at least about 5 μg), zinc (at least about 0.5 mg), copper (at least about 60 μg), manganese (at least about 5 μg), sodium (from about 20 to about 60 mg), potassium (from about 80 to about 200 mg), chloride (from about 55 to about 150 mg) and selenium (at least about 0.5 mcg).

The infant formulas may further comprise fructopolysaccharides, concentrations of which may range up to about 5% by weight of the formula, on an as fed basis, including from about 0.05% to about 3%, and also including from about 0.1% to about 2%. These fructopolysaccharides may be long chain (e.g., inulin), short chain (e.g., FOS or fructooligosaccharides), or combinations thereof with mixtures comprising varied chain length structures, most of which have a DP (degree polymerization) of from about 2 to about 60.

The infant formulas may further comprise other optional ingredients that may modify the physical, chemical, aesthetic or processing characteristics of the compositions or serve as pharmaceutical or additional nutritional components when used in the targeted infant or infant population. Many such optional ingredients are known or are otherwise suitable for use in nutritional products and may also be used in the infant formulas of the present invention, provided that such optional materials are compatible with the essential materials described herein and are otherwise suitable for use in an infant formula.

Non-limiting examples of such optional ingredients include additional anti-oxidants, emulsifying agents, buffers, colorants, flavors, lactoferrin, additional alpha lactalbumen, nucleotides and nucleosides, probiotics, prebiotics, and related derivatives, thickening agents and stabilizers, and so forth.

### Method of Use

The present invention is also directed to a method of accelerating brain development in an infant, by preparing the infant formulas as described herein and then administering or instructing a caregiver to administer the formula to an infant during the first 2 months, preferably during the first 4 months, of life.

The present invention is also directed to a method of accelerating neural migration in an infant, by preparing the infant formulas as described herein and then administering or instructing a caregiver to administer the formula to an infant during the first 2 months: preferably during the first 4 months, of life.

The present invention is also directed to a method of accelerating vision development in an infant, by preparing the infant formulas as described herein and then administering or instructing a caregiver to administer the formula to an infant during the first 2 months, preferably during the first 4 months, of life.

The present invention is also directed to a method of accelerating cognitive development in an infant, by preparing the infant formulas as described herein and then administering or instructing a caregiver to administer the formula to an infant during the first 2 months, preferably during the first 4 months, of life.

The present invention is also directed to a method of providing sole source, supplement, or primary nutrition to an infant, by preparing the infant formulas as described herein and then administering or instructing a caregiver to administer the formula to an infant during the first 2 months, preferably during the first 4 months, of life.

All of the methods of the present invention are directed to the selected use of the infant formulas during the first 2-4 months of life, although it is understood that such methods may include additional administration, so that after the initial 2-4 month period the infant continues to feed on the same formula for up to 9-12 months. To realize the benefits of the present invention, however, administration must still occur during the first 2-4 months of life, even if such administration extends well beyond that period of time.

In the context of the methods of the present invention, the infant formulas may provide sole, primary, or supplemental nutrition, although sole source nutrition is preferred. For powder embodiments, each method may also include the step of reconstituting the powder (or instructing a caregiver to reconstitute) with an aqueous vehicle, most typically water or human milk, to form the desired caloric density, which is then orally or enterally fed to the infant to provide the desired nutrition. The powder is reconstituted with a quantity of water, or other suitable fluid such as human milk, to produce a volume and nutrition profile suitable for about one feeding.

The infant formulas of the present invention have a caloric density that most typically ranges from about 19 to about 24 kcal/fl oz, more typically from about 20 to about 21 kcal/fl oz, on an as fed basis.

### Ganglioside Analytical Method

Ganglioside concentrations for use herein are determined in accordance with the following analytical method.

Total lipids are extracted from Lacprodan MFGM-10 or infant formula samples with a mixture of chloroform:methanol:water. Gangliosides are purified from the total lipid extract by a combination of diisopropyl ether (DIPE)/1-butanol/aqueous phase partition and solid phase extraction through C-18 cartridges. Lipid-bound sialic acid (LBSA) in the purified gangliosides is measured spectrophotometrically by reaction with resorcinol. The amount of gangliosides in the samples is obtained by multiplying LBSA by a conversion factor. This factor is obtained from the molecular weight ratio of gangliosides and sialic acid units. Because gangliosides are a family of compounds with different molecular weights and number of sialic acid residues, HPLC separation is used to measure individual ganglioside distribution in order to calculate this conversion factor more accurately.

Standards
Disialoganglioside GD1a, from bovine brain, min. 95% (TLC) SIGMA, ref G-2392.
Monosialoganglioside GM1, from bovine brain, min. 95% (TLC) SIGMA, ref G-7641.
Disialoganglioside GD3 ammonium salt, from bovine buttermilk, min. 98% (TLC) Calbiochem, ref 345752 or Matreya, ref. 1503.
Monosialoganglioside GM3 ammonium salt from bovine milk, min. 98% (TLC) Calbiochem, ref 345733 or Matreya, ref. 1504.
N-acetylneuraminic acid, (sialic acid, NANA) from Escherichia coli, min. 98% SIGMA, ref A-2388.

Ganglioside standards are not considered as true standards since suppliers don't typically guarantee their concentrations. For this reason, concentrations are estimated as LBSA measured by the resorcinol procedure. The standards are diluted with chloroform:methanol (C:M)1:1 (v/v) to a theoretical concentration of 1-2.5 mg/ml depending on the type of ganglioside. Aliquots of 10, 20 and 40 μl are taken, brought to dryness under N2 stream and measured as explained below (Measurement of LBSA). An average concentration of the three aliquots is considered as concentration of ganglioside standards expressed as LBSA. Ganglioside concentration is obtained by multiplying LBSA by a conversion factor obtained from molecular weight ratios (Conversion factor:

$$\frac{Ganglioside M W}{n \times Sialic acid M W}$$

where n=number of sialic acid units).

| | |
|---|---|
| Reagents | |
| Chloroform, HPLC grade, Prolabo. | Sodium dibasic phosphate, PA, Panreac. |
| Methanol, HPLC grade, Merck. | Hydrochloric acid 35%, PA, Panreac. |
| Diisopropyl ether, HPLC grade, Prolabo. | Copper sulphate, PA, Panreac. |
| Butyl acetate, PA, Merck. | Resorcinol, 99%, Merck. |
| 1-Butanol, PA, Merck. | Sodium chloride, PA, Panreac. |
| Equipment | |
| Analytical balance, with a precision of 0.1 mg. | Centrifuge |
| HPLC vials, screws cap and inserts from Waters. | Ultrasonic bath |
| Micro syringes Hamilton (50, 100, 250, 500, 1000 μl). | SPE-Vacuum manifolds 24-port |
| HPLC: Alliance 2690 from Waters. | model |
| HPLC UV Detector, reference number 2487, | Diaphragm vacuum pump |
| from Waters. | Triple-Block Reacti-Therm III |
| HPLC Integrator: Waters Millennium 32. | (Pierce) |
| Solvac Filter Holder (polypropylene), ref No. 4020. | Water-vacuum pump |
| Durapore membrane filters of 0.45 μm, | Glass Pasteur pipette |
| ref. No. VLP04700 | Organic solvent dispenser (2.5-25 ml) |
| Multi-reax Vortex (Heidoiph) | Vortex (Heidolph) |
| Digital pipettes (2-20, 5-50, 40-200, 200-1000 μl) | Water bath 40-100° C. |
| Glass round-bottom 10 ml centrifuge tube | Glass pipettes (5, 10, 25 ml). |
| Glass round-bottom 50 ml centrifuge tube | Spectrophotometer |
| Class conic-bottom 40 ml centrifuge tube | (ThermoSpectronic UV500). |
| 500 mg C-18 cartridges (5 ml, ref 52604-U, Supelco) | |
| Reacti-Vap III evaporator 27-port model (Pierce) | |

### Procedure

Lipid Extraction: lipid extracts are prepared as follows: samples of 1 g of formula or 100 mg of Lacprodan MFGM-10 are weighed into round-bottom glass centrifuge tubes (50 ml tubes for formula and 10 ml tubes for Lacprodan MFGM-10). Twenty-five ml chloroform:methanol:water (C:M:W) 50:50:10 (v/v) per g of sample are added, being samples completely dispersed by alternative vortexing and sonication for 1 min. Tubes are incubated for 45 min at room temperature with vigorous and continuous vortexing (2000 rpm) with bath sonication pulses of 1 min every 15 min. Samples are centrifuged (1500×g, 10 min, 15° C.). The supernatants are transferred to 40 ml conical-bottom glass centrifuge tubes and started to bring to dryness under N2 at 37° C. Meanwhile, the pellets are reextracted with 12.5 ml of C:M:W per g for 15 min at room temperature with continuous vortexing (2000 rpm) and with bath sonication pulses of 1 min every 7.5 min. After centrifugation, the supernatants are pooled with the first ones in the 40 ml tubes and the evaporation continued. The pellets are washed with C:M 1:1 (v/v) and incubated 10 min in the same conditions than before, with sonication pulses every 5 min. After centrifugation, the supernatants are also added to the 40 ml tubes and evaporated.

The ganglioside fraction is purified from the total lipid extract by a combination of the diisopropyl ether (DIPE)/1-butanol/aqueous phase partition described by Ladisch S. and Gillard B. (1985) A solvent partition method for microscale ganglioside purification, Anal. Biochem, 146:220-231. This is followed by solid phase extraction through C-18 cartridges as described by Williams M and McCluer R (1980), The use of SeoPak™ C18 cartridges during the isolation of gangliosides, J. Neurochem, 35:266-269 with modifications.

Diisopropyl ether/1-Butanol/Aqueous NaCl partition: 4 ml of DIPE/1-butanol 60:40 (v/v) are added to the dried lipid extract. Samples are vortexed and sonicated to achieve fine suspension of the lipid extract Two ml of 0.1% aqueous NaCl are added, and the tubes alternately vortexed and sonicated for 15 second pulses during 2 min, and then centrifuged (1500×g, 10 min, 15° C.). The upper organic phase (containing the neutral lipids and phospholipids) is carefully removed using a Pasteur pipette taking care of not removing the interphase. The lower-aqueous phase containing gangliosides is extracted twice with the original volume of fresh organic solvent. The samples are partially evaporated under a stream of N2 at 37° C. during 30-45 min until the volume (nearly 2 ml) is reduced to approximately one half of the original volume.

Solid Phase Extraction (SPE) through reversed-phase C-18 cartridges: 500 mg C-18 cartridges are fitted to a twenty four-port liner SPE vacuum manifold and activated with three consecutive washes of 5 ml of methanol, 5 ml of C:M 2:1 (v/v) and 2.5 ml of methanol. Then, cartridges are equilibrated with 2.5 ml of 0.1% aqueous NaCl:methanol 60:40 (v/v). The volumes of partially evaporated lower phases are measured, brought up to 1,2 ml with water, and added with 0.8 ml methanol. Then, they are centrifuged (1500×g, 10 min) to remove any insoluble material and loaded twice onto C-18 cartridges. SPE cartridges are swished with 10 ml of distilled water to remove salts and water-soluble contaminants and then, dried 30 seconds under vacuum. Gangliosides are eluted with 5 ml of methanol and 5 ml of C:M 2:1 (v/v), dried under a stream of N2 and redissolved in 2 ml of C:M 1:1 (v/v). Samples and solvents are passed through the cartridges by gravity or forced by weak vacuum with a flow rate of 1-1.5 ml/min. Gangliosides are stored at −30° C. until analysis. Total gangliosides are measured as LBSA. An aliquot of 500 μl is placed into a 10 ml glass centrifuge tube, dried under N2, and measured by resorcinol assay (3).

Measurement of LBSA: 1 ml of the resorcinol reagent and 1 ml of water are added. The tubes are cupped and heated for 15 min at 100° C. in a boiling water bath. After heating, the tubes are cooled in a icebath water, 2ml of butyl acetate:butanol 85:15 (v/v) are added, the tubes are sacked vigorously for 1 min and then centrifuged at 750×g for 10 min. The upper phases are taken and measured at 580 nm in a spectrophotometer Standard solutions of NANA (0, 2, 4, 8, 16, 32 and 64 μg/ml) are treated the same way and are used to calculate the sialic acid concentration in samples.

The resorcinol reagent is prepared as follows: 10 ml of resorcinol at 2% in deionised water, 0.25 ml of 0.1 M copper sulphate, 80 ml of concentrated hydrochloric acid, complete up to 100 ml with water. The reagent is prepared daily protected from light.

Separation of gangliosides by HPLC: gangliosides are separated by HPLC in a Alliance 2690 equipment with Dual Absorbance Detector, from Waters using a Luna-NH2 column, 5 μm, 100 Å, 250×4.6 mm from Phenomenex, ref. 00G4378-EO. They are eluted at room temperature with the following solvent system: acetonitrile-phosphate buffer at different volume ratios and ionic strengths according to the method of Gazzotti G., Sonnion S., Ghidonia R (1985), Normal-phase high-performance liquid chromatographic separation of non-derivatized ganglioside mixtures. J Chromatogr. 348:371-378.

A gradient with two mobile phases is used;

Solvent A: Acetonitrile—5 mM phosphate buffer, pH 5.6 (83:17). This buffer is prepared with 0.6899 g NaH2PO4.H2O to 1 L water, pH adjusted to 5.6

Solvent B: Acetonitrile—20 mM phosphate buffer, pH 5-6 (1:1). This buffer is prepared with 2.7560 g NaH2PO4.H2O to 1 L water, pH adjusted to 5.6

The following gradient elution program is used:

| | Flow | | |
|---|---|---|---|
| Time (min) | (ml/min) | % A | % B |
| 0 | 1 | 100 | 0 |
| 7 | 1 | 100 | 0 |
| 60 | 1 | 66 | 34 |
| 61 | 1 | 0 | 100 |
| 71 | 1 | 0 | 100 |
| 72 | 1 | 100 | 0 |
| 85 | 1 | 100 | 0 |

Samples are liquid-phase extracted, partitioned and solid-phase extracted as explained above. An aliquot of 0.5 ml from the 2 ml sample in C:M 1:1 is evaporated under nitrogen and redissolved into 0.150 ml of water. For perfect reconstitution, the sample is vortexed and sonicated. The final solution is transferred to an HPLC vial. The injection volume is 30 μl for samples and standards.

GD3 and GM3 standards are measured by the resorcinol procedure and true concentrations calculated as explained above. Four standard solutions containing GD3 and GM3, and a blank are prepared in water. The concentrations of the calibration standards ranged approximately from 0-0.5 mg/ml for GD3 and from 0-0.2 mg/ml for GM3. The exact concentration of each set of standards may vary depending on the purity of the standards.

A set of standards is injected each time the system is set-up, e.g., for a new column. The proper performance of the system is checked by injecting one standard of intermediate concentration every ten runs. If the interpolated concentration is not between 95%-105% of the theoretical concentration, a new calibration set is injected and used for subsequent calculations.

### Method of Manufacture

The infant formulas of the present invention may be prepared by any known or otherwise effective technique, suitable for making and formulating infant or similar other formulas. Such techniques and variations thereof for any given formula are easily determined and applied by one of ordinary skill in the infant nutrition formulation or manufacturing arts in the preparation of the formulas described herein.

Methods of manufacturing the infant formulas of the present invention may include formation of a slurry from one or more solutions which may contain water and one or more of the following: carbohydrates, proteins, lipids, stabilizers, vitamins and minerals. This slurry is emulsified, homogenized and cooled. Various other solutions, mixtures or other materials may be added to the resulting emulsion before, during, or after further processing. This emulsion may then be further diluted, sterilized, and packaged to form a ready-to-feed or concentrated liquid, or it can be sterilized and subsequently processed and packaged as a reconstitutable powder (e.g., spray dried, dry mixed, agglomerated).

Other suitable methods for making infant formulas are described, for example, in U.S. Pat. No. 6,365,218 (Borschel) and U.S. Patent Application 20030118703 A1 (Nguyen, et al.), which descriptions are incorporated herein by reference.

### EXPERIMENT 1

The purpose of this study is to compare the performance benefits in neonatal pigs fed either a control formula or one of two different formulas with enriched concentrations of gangliosides, phospholipids, and sialic acid, and varied concentrations of arachidonic and docosahexaenoic acids.

### Background

The neonatal piglet constitutes an appropriate model to evaluate nutritional intervention prior to the design and implementation of human clinical trials. Its suitability resides in the similarities of the gastrointestinal physiology of the piglet to that of the human neonate (Miller, E. R., Ullrey, The pig as model for human nutrition, Annu Rev Nutr 1987; 7; 361-82). In addition, piglet brain growth spurt, like that of human, extends from late prenatal to early postnatal life, which also constitutes a great advantage of this animal model (Pond W G et al. Perinatal Ontogeny of Brain Growth in the Domestic Pig. PSEBM 2000, 223:102-108). The critical period to consider is 70 through 140 days postconception (birth takes place around 112-113 days postconception). The present study is designed to provide a biological assessment of the effects of three test formulas, one of which is a conventional infant formula control.

### Summary

The data from the study show significant neural migration at 12-13 days of age in the neonatal piglets. This time period in the piglet would correspond to between 3 and 4 months in a human infant. (Miller, E. R., Ullrey, The pig as model for human nutrition, Annu Rev Nutr 1987; 7; 361-82).

### Experimental Design

The study is longitudinal and includes 3 groups of piglets fed the experimental diets, A, B or C (see Table 2) with three time points of sacrifice after 89, 15-16 and 29-30 days of feeding. An additional group, sacrificed at the beginning of the study, is used as a reference. The study is divided into two experiments. Piglets in the study are supplied by a certified farm.

In the first of two experiments in the study, 33 male domestic piglets (4-5day old) are housed in stainless steels wire cages (2 animals per cage) in a conditioned room at 27-30° C. The animals are fed 4 times a day with an adapted pig diet, according to their nutritional requirements. After an adaptation period of 3 days, 3 piglets are sacrificed. The time at which these animals are sacrificed is considered “Time Zero” in the study. The rest of the piglets are paired by weight and litter, and are divided into 3 groups (n=10, n=10, and n=10, respectively) that are fed also 4 times a day with the following diets:
Diet A: Similar to Similac® Advance® Infant Formula, available from Abbott Laboratories, Columbus, Ohio USA (0.4% arachidonic acid, 0.15% docosahexaenoic acid, by weight of total fatty acids, and conventional whey protein concentrate).
Diet B: Infant formula of the present invention with 0.4% arachidonic and 0.15% docosahexaenoic acid, by weight of total formula fatty acids, and enriched whey protein concentrate at a level of 7.1 g/L of formula on an as-fed basis.
Diet C: Infant formula similar to Diet B but with reduced arachidonic and docosahexaenoic acid concentrations (0.2% and 0.1%, respectively, by weight of total formula fatty acids) and enriched whey protein concentrate at a level of 7.1 g/L of formula on an as-fed basis.

Diets A, B and C are adapted in terms of micronutrients (minerals and vitamins) to the special requirements of neonatal piglets. The following table shows the composition of the standard pig diet and of diets A, B and C.

TABLE 2
Experimental Diets

| | Standard pig | Standard | Diets A, | Diets A, |
|---|---|---|---|---|
| | diet | pig diet | B, C per | B, C per |
| | per 100 g | 100 ml | per 100 g | 100 ml |
| Protein | 25.5 | 4.79 | 10.9 | 1.40 |
| Fat | 36.3 | 6.82 | 28.9 | 3.71 |
| Carbohydrates | 31 | 5.83 | 53 | 6.81 |
| Ash | 5.2 | 0.98 | 5.2 | 0.67 |
| Moisture | 2 | 0.38 | 2 | 0.26 |
| Minerals | | | | |
| Na (mg) | 201.9 | 37.96 | 201.9 | 25.94 |
| K (mg) | 800 | 150.40 | 800 | 102.80 |
| Cl (mg) | 300 | 56.40 | 300 | 38.55 |
| Fe (mg) | 32.7 | 6.15 | 32.7 | 4.20 |
| Zn (mg) | 13 | 2.44 | 13 | 1.67 |
| Cu (mg) | 0.8 | 0.15 | 0.8 | 0.10 |
| Mg (mg) | 61.4 | 11.54 | 61.4 | 7.89 |
| Mn (mg) | 0.5 | 0.09 | 0.5 | 0.06 |
| Ca (mg) | 1069 | 200.97 | 1069 | 137.37 |
| P (mg) | 792 | 148.90 | 792 | 101.77 |
| I (mcg) | 61.7 | 11.60 | 61.7 | 7.93 |
| Se (mcg) | 20 | 3.76 | 20 | 2.57 |
| Vitamins | | | | |
| Vitamin A (IU) | 400 | 75.20 | 400 | 51.40 |
| Vitamin D (IU) | 53 | 9.96 | 53 | 6.81 |
| Vitamin E (IU) | 5 | 0.94 | 5 | 0.64 |
| Vitamin K (mcg) | 21.5 | 4.04 | 21.5 | 2.76 |
| Thiamine (mg) | 0.2 | 0.04 | 0.2 | 0.03 |
| Riboflavin (mg) | 0.5 | 0.09 | 0.5 | 0.06 |
| Pyridoxine (mg) | 0.317 | 0.06 | 0.317 | 0.04 |
| Cyanocobalamine | 3.5 | 0.66 | 3.5 | 0.45 |
| (mcg) | | | | |
| Pantothenic acid (mg) | 2 | 0.38 | 2 | 0.26 |
| Folic acid (mcg) | 100 | 18.80 | 100 | 12.85 |
| Biotin (mcg) | 26.5 | 4.98 | 26.5 | 3.41 |
| Niacin (mg) | 3 | 0.56 | 3 | 0.39 |
| Vitamin C (mg) | 71.25 | 13.40 | 71.25 | 9.16 |
| Choline (mg) | 170 | 31.96 | 170 | 21.85 |
| Others | | | | |
| Nucleotides (mg) | — | — | 56.14 | 7.21 |
| Energy | 552.7 | 103.91 | 515.7 | 66.27 |

TABLE 3

| | | Diet B | Diet C |
|---|---|---|---|
| | Diet A | PSNU | PSNU 29002 |
| | (control) | 29002 (7.1 | (7.1 g/L |
| Protein | Milacteal-651 | g/L as-fed) | as-fed) |
| Ganglioside mg/L | 3.2-4.8 | 14 | 14 |
| Sialic acid mg/L | 115-150 | 190 | 190 |
| Lipid-bound sialic acid | <0.1% | 2.5-3.0 | 2.5-3.0 |
| (wt % of total sialic acid) | | | |
| Phospholipid mg/L | 118 | 450 | 450 |
| Lactoferrin mg/L | 2.6 | 100 | 100 |
| FOS g/L | 0 | 2 | 2 |
| Arachidonic acid - wt % | 0.4 | 0.4 | 0.2 |
| of total fatty acids | | | |
| Docosahexaenoic acid - wt | 0.15 | 0.15 | 0.1 |
| % of total fatty acids | | | |

All diets, once prepared, are used immediately or are stored in inert atmosphere cans at 4° C. and used within 24 hours. Diets are in powder form and are reconstituted with water to 18.8% by weight for the adapted pig diet and to 12.85% by weight for Diets A, B, and C. The reconstituted liquid diets are poured on the cage feeders. The remaining liquid is removed and measured and the feeders are cleaned prior to subsequent feedings.

For each group, 3 or 4 piglets are sacrificed at 89, 15-16 and 29-30 days after the initiation of feeding with control (Diet A) or experimental formulas (Diets B and C).

In the second experiment of the study, 44 male domestic piglets (4-5-day old) are housed individually in the same type of cages and in the same room described for the first experiment. The feeding protocol is the same and 4 piglets are sacrificed, after the adaptive period, to complete the reference group. The rest of the piglets are paired by weight and litter and divided into 3 groups (n=13, n=13, and n=14, respectively) that are fed with diets A, B and C. One or two piglets more are included on each group to replace withdrawals.

Dietary intake and weight gain are monitored 4 times a day twice weekly, respectively, for each piglet.

At the appropriate time, each piglet is anaesthetized with Ketamine/Domtor after overnight fasting and then sacrificed by jugular puncture terminal bleeding. The composition and histology of the brain is subsequently evaluated.

### Sample Preparation

Piglets are deprived of food overnight and bled to death via jugular vein puncture while under anesthesia. Blood is collected with tripotassium EDTA (2.7 mmol/L) as anticoagulant and centrifuged at 1500×g for 10 min at 4° C.

Skulls are opened and brains removed and weighed. The left hemisphere is dissected and immersed in buffered 4% formaldehyde pH 7.4 and in ethanol at 70° for one week for histological analysis. The right hemisphere is stored at −80° C. for biochemical analysis. Whole eyes are removed. The left eye is also immersed in formaldehyde. Two hours later the anterior pole of the eye is separated with a scalpel and the eye kept again in formaldehyde for 18 h. The right eye is dissected and the retina removed and weighed. Plasma, right hemisphere and retina are stored at −80° C. until analysis.

### Fatty Acid Composition of Plasma

Plasma samples are methylated by the method of Lepage and Roy (6) and analyzed by gas-liquid chromatography. Two hundred microliters (μL) of plasma are added with pentadecanoic acid as internal standard (0.04 mg/sample), 2 ml of a mixture of methanol:hexane (4:1) and 0.2 ml acetyl chloride. Tubes are capped and heated at 100° C. for 1 hour. They are then cooled in an ice bath and added with 5 ml 6% K2CO3, and centrifuged for 10 min at 1500×g. Three microliters of the hexane upper layer are injected into a Hewlett-Packard 6890 chromatograph equipped with flame ionization detector and 60 m long, 0.32 mm id, 0.2 μm film thickness capillary SP2330 column (Supelco). Helium flow rate 1 ml/min is used as carrier gas with split ratio 1:40. Temperature programming consisted of 165° C. for 3 min, increase of 2° C./min to 195° C., held 2 min, increase of 3° C./min to 211° C., held 10 min. Injector and detector temperatures are 250° C. Fatty acids are identified by comparing their retention times with those of authentic standards (Sigma). Results are expressed as normalized percentages of area or concentrations for each fatty acid methyl ester.

### Brain Composition

The right hemisphere is homogenized in a Heidolph homogenizer. One gram of the homogenized cerebrum is further homogenized with 15 ml PBS in ultraturrax for 1 min and diluted to 100 ml with PBS. The content of DNA is measured in 10 μL aliquots, in triplicate, by reaction with the Hoechst reagent and fluorimetry using the Molecular Probes kit F-2962.

Protein content is determined in a 1:4 dilution of the 1 g/100 ml homogenate by the Lowry procedure using the Sigma kit TP0300 with modifications to measure in microplates Briefly, 20 □l of samples or standards, in triplicate, are placed in 96-well microplates. Eighty μl water, and 100 μl Lowry reagent are added and incubated for 20 min with mixing. Fifty μl of Folin-Ciocalteau reagent are added and incubated for 30 min with mixing. Absorbance is measured at 690 nm.

Cholesterol is measured by spectrophotometric-colorimetric method after extraction of sample with organic solvents. Two hundred mg of the homogenized brain are further homogenized in 1 ml water in Heidolph homogenizer. Samples are added with 5 ml hexane:isopropanol (3:2), vortexed for 1 min, sonicated for 5 min, and centrifuged for at 4° C. for 10 min at 1500×g. The upper layer is collected and the lower layer is reextracted with 3 ml solvents. The upper layer is collected, pooled with the first one and evaporated under N2 stream. The extract is dissolved in 3 ml chloroform, and 20 μl are taken in duplicate for cholesterol analysis. The solvent is evaporated and 100 μl of isopropanol are added. Cholesterol determination is done using the Randox kit n° CH201 according to the supplier instructions. Cholesterol calibration line is used from 0.25 to 2 mg/ml.

Fatty acid composition is measured as explained above for plasma, using 40 mg of homogenate and without internal standard. Results are expressed as normalized percentages of area for each fatty acid methyl ester.

Ganglioside content is measured both by HPLC and by spectrophotometry as lipid-bound sialic acid (LBSA) after extraction, partition and purification of lipids. A portion of homogenized brain (1.250 g) is extracted with 18 ml chloroform:methanol (C:M) 1:1 (v/v); the mixture is stirred for 45 min at 4° C. and centrifuged at 1500×g for 10 minutes at 4° C. The supernatant is colleted and the pellet reextracted twice with 18 ml and 12 ml solvent mixture, respectively. The three supernatants are pooled and brought to 50 ml with solvent mixture, and two aliquots of 20 ml are taken and incubated overnight at −30° C. After incubation, the samples are centrifuged and the supernatants collected and desiccated under N2 stream. Gangliosides are purified from the total lipid extract by a combination of the diisopropyl ether (DIPE)/1-butanol/aqueous phase partition (described by Ladisch and Gillard, 1985, A solvent partition method for microscale ganglioside purification, Anal. Biochem., 46:220-231) followed by solid phase extraction through C-18 cartridges (according to the method of Williams and McCluer, 1980, The use of Sep-Pak™ C18 cartridges during the isolation of gangliosides, J. Neurochem. 35:266-269) with modifications.

Four ml of DIPE/1-butanol 60:40 (v/v) are added to the dried lipid extracts. Samples are vortexed and sonicated to achieve fine suspension of lipids. Two ml 0.3% aqueous NaCl are added, and the tubes alternately vortexed and sonicated for 15 second pulses during 2 min, and then centrifuged. The upper organic phase (containing neutral lipids and phospholipids) is carefully removed using a Pasteur pipette taking care of not removing the interphase. The lower-aqueous phase containing gangliosides is extracted twice with the original volume of fresh organic solvent. The samples are partially evaporated under N2 stream at 37° C. during 30-45 min, until volume (nearly 2 ml) is reduced to approximately one half of the original volume.

Five hundred mg C-18 cartridges are fitted to a twenty four-port liner SPE vacuum manifold and activated with three consecutive dishes of 5 ml methanol, 5 ml C:M 2:1 (v/v) and 2.5 ml methanol. Then, cartridges are equilibrated with 2.5 ml 0.3% aqueous NaCl:methanol 60:40 (v/v). The volumes of partially evaporated lower phases are measured, brought up to 1,2 ml with water, and added with 0.8 ml methanol. Then, they are centrifuged to remove any insoluble material and loaded twice onto C-18 cartridges. SPE cartridges are finished with 10 ml distilled water to remove salts and water-soluble contaminants and then, dried 30 seconds under vacuum. Gangliosides are eluted with 5 ml methanol and 5 ml C:M 2:1 (v/v), dried under N2 stream and redissolved in 1 ml C:M 1:1 (v/v). Total gangliosides are measured as LBSA. An aliquot of 50 μl is placed into 10 ml glass centrifuge tube, dried under N2, and measured by resorcinol assay (Svennerholm, L., 1957, Quantitative estimation of sialic acid: A colormetric resorcinol-hydrochloric acid method, Biochem. Biophys. Acta., 24:604-611).

One ml of the resorcinol reagent and 1 ml of water are added. The tubes are cupped and heated for 15 min at 100° C. in boiling water bath. After heating, the tubes are cooled in ice-bath water, and 2 ml butyl acetate:butanol 85:15 (v/v) are added. Tubes are shaked vigorously for 1 min and then centrifuged at 750×g for 10 min. The upper phases are taken and measured at 580 nm in a spectrophotometer. Standard solutions of NANA from 2-64 □g/ml are treated the same way and are used to calculate sialic acid concentration in samples.

The resorcinol reagent is prepared as follows: 10 ml of resorcinol at 2% in deionised water, 0.25 ml of 0.1 M copper sulphate, 80 ml of concentrated hydrochloric acid, complete up to 100 ml with water. The reagent is prepared daily and protected from light.

One hundred and fifty mcg of the rest of the purified lipid extract is used for ganglioside analysis by HPLC. Gangliosides are separated by HPLC in Alliance 2690 equipment with Dual Absorbance Detector, from Waters, using a Luna-NH2 column, 5 μm, 100 Å, 250×4.6 mm from Phenomenex.

They are eluted at room temperature with the following solvent system: acetonitrile-phosphate buffer at different volume ratios and ionic strengths (according to the method of Gazzotti, Sonnino, and Ghidoni, 1985, Normal-phase high-performance liquid chromatographic separation of non-derivitized ganglioside mixtures, J. Chromotogr., 348:371-378).

A gradient with two mobile phases is used:

Solvent A: Acetonitrile—5 mM phosphate buffer, pH 5.6 (83:17)

Solvent B: Acetonitrile—20 mM phosphate buffer, pH 5.6 (1:1).

The following gradient elution program is used:

| | Flow | | |
|---|---|---|---|
| Time (min) | (ml/min) | % A | % B |
| 0 | 1 | 100 | 0 |
| 7 | 1 | 100 | 0 |
| 60 | 1 | 66 | 34 |
| 80 | 1 | 36 | 64 |
| 81 | 1 | 0 | 100 |
| 90 | 1 | 0 | 100 |
| 91 | 1 | 100 | 0 |
| 105 | 1 | 100 | 0 |

GD3 solutions from 0-0.4 mg/ml are used as calibration standards and bovine brain solution is used to identify ganglioside classes.

### Retina Composition

Retina a homogenized with 3.5 ml C:M 1:1 (v/v) in ultraturrax for 1 min, vortexed for 45 minutes and centrifuged. The supernatant is collected and the pellets reextracted twice with 2 ml solvent mixture. The three supernatants are pooled and desiccated under N2. The extracts are dissolved in 1 ml chloroform and 100 pi aliquots are taken for analysis of fatty acids and phospholipids. The rest of the extract is desiccated again and subjected to the same partition and purification procedure than brain samples. The purified extracts are dissolved in 1 ml C:M 1:1, 0.5 ml are measured by resorcino procedure and 0.5 ml are used for ganglioside analysis by HPLC.

Fatty acid composition is measured in the 100 μl aliquots as explained above for plasma. Results are expressed as normalized percentages of area for each fatty acid methyl ester.

Phospholipid content of retina samples is measured by HPLC in an Spherisorb silica column, 5 μm, 150×4.6 mm using the following solvent system: acetonitrile-phosphate buffer at different volume ratios and ionic strengths.

A gradient with two mobile phases is used:

Solvent A: Acetonitrile

Solvent B: Acetonitrile—5 mM phosphate buffer, pH 5 (80:20).

The following gradient elution program is used with column working at 55° C.:

| | Flow | | |
|---|---|---|---|
| Time (min) | (ml/min) | % A | % B |
| 0 | 2 | 95 | 5 |
| 2 | 2 | 95 | 5 |
| 5 | 2 | 70 | 30 |
| 12 | 2 | 10 | 90 |
| 20 | 2 | 95 | 95 |

Twenty μl of the 100 μl aliquot are injected into the system (Alliance 2690 with Dual Absorbance Detector, from Waters). The detection is done at 201 nm. Multicompound calibration standards of phosphatidylserine (PS), phosphatidylethanolamine (PE), phosphatidylcholine (PC), and sphyngomyelin (SM) are used from 0.2-5 mg/ml. Phosphatidylinositol is injected separately because it contained PE as contaminant. The same range of concentrations is used.

### Histologic Analysis of Brain and Eye

Brian hemispheres are transversely sectioned into 50-mm thick specimens. After a preliminary analysis, central blocks (4, 5 or 6 according to brain size) are selected for the quantifications.

A sample of the optic nerve with minimum length of 5 mm is transversely sectioned, fixed in buffered formalin for 3 h and then preserved in phosphate buffer (pH 7.4) at 4-6° C.

The eyes are frontally sectioned into 3 specimens, labeled and embedded in paraffin. Serial sections are made of all paraffin blocks for subsequent staining.

After serial sectioning on a microtome and mounting on normal and special slides for immunohistochemical procedures, they are stained with the classic staining: Haematoxylin-Eosin, Periodic Acid Schiff (PAS) Reaction, and Klüver-Barrera Luxol Fast Blue. Immunohistochemical staining is also performed on histologic sections from the same series used for classic staining. The following markers are used:

Monoclonal antibody S100 Protein Ab-1. S100 belongs to the family of calcium binding proteins such as calmodulin and troponin C. S100 protein is also expressed in the antigen presenting cells such as the Langerhans cells in skin and interdigitating reticulum cells in the paracortex of lymph nodes and stains astroglia cells. The immunogen used is purified bovine brain S100 protein (species reactivity: human, cow, rat, and mouse).

Monoclonal antibody anti-neural nuclei (NeuN). NeuN (or Neuronal Nuclei) reacts with most neuronal cell types. Developmentally, immunoreactivity is first observed shortly after neurons have become postmitotic; no staining has been observed in proliferative zones. The immunohistochemical staining is primarily localized in the nucleus of the neurons with lighter staining in the cytoplasm. Species reactivity: human, mouse, rat, pig, ferret, chick and salamander.

Monoclonal antibody bcl-2. Expression of bcl-2alpha oncoprotein inhibits programmed cell death (apoptosis). Species reactivity: human and pig.

Thirty images of subcallosal fasciculus and others from adjacent white matter are captured with a black and white Sony XC-ST500CE video camera (Sony Corporation, Tokyo, Japan) coupled to an Olympus BH-2 microscope (20 watt) with MTV-3 adapter (Olympus Optical Company, Ltd., Tokyo, Japan). Use of 20× and 60× power objectives Olympus PLCN60× (60×/0.80) yielded a total magnification of 600×. The image processing is done using Visilog 6.0 software (Noesis S.A. Courteboeutf France).

### Results

### Withdrawals

Experiment 1: One piglet from group A is very small at birth and does not catch up with the rest of the piglets. One pig of group C dies 10 days after enrolment. Another pig of group C is a female, which is confirmed at the end of the experiment. Consequently, n for group A at 29-30 days is 3 instead of 4, and n of group C at the same age is 2 instead of 4.

Experiment 2: One piglet dies during the period of adaptation. Another piglet of group B dies 6 days after enrolment. Two pigs of group A and one in group B are excluded of the study, because they are very small at birth and did not grow as the rest of piglets.

Consequently, the complete study target of 7 piglets for each time point and group is met in all of the groups except for group A at 29-30 days (n=6).

### Body Weight and Dietary Intake

The evolution of body weight and dietary intake is very similar for the 3 different dietary groups. There are no differences in body weight evolution among groups for the duration of the experiment. Dietary intake is significantly higher in group C than in groups A and B, only for the interval of time between 16 and 28 days. For the rest of the time there are no differences among groups. When the intake is represented as accumulated dietary intake there are no differences among groups. Likewise, the evolution of the food efficiency, calculated as grams of body weight/100 kcal of intake is similar for the 3 groups. There are no differences among the groups when different intervals of time are considered or for the entire study period.

### Fatty Acid Composition of Plasma

All fatty acids tended to decrease at 89 days and then increased over time until 29-30 days of feeding. This is likely due to lower intake of formula during the first week of study due to the incidence of diarrhea and/or adaptation issues. Regarding long-chain polyunsaturated fatty acids, there are no significant differences among groups at each time point However, group C had the lowest concentration of these fatty acids at the end of the study resembling the composition of the formula.

### Brain Composition

The contents of protein, DNA and cholesterol in brain are measured as indexes of protein mass, cell number (DNA) and myelinization (cholesterol). There are no significant differences among groups at any time point. However, there are some evidences that can be concluded from the data. The amount of DNA did not increase in brain whereas protein tended to increase indicating that cell density in brain is similar in piglets during the period of study and that cell multiplication occurs as a consequence of brain growth. Cholesterol increased both per gram of tissue and when considering total brain, which means that myelinization takes place at least during the period of study considered in the experimental design.

Regarding fatty acid composition, there are no significant differences among groups for any fatty acid concentrations at any time point. There are some trends over time for the study groups: decrease of 16:0 and 20:4n-6 and increase of dimethyl acetals, 18:1 n-9, and 18:2n-6.

Total ganglioside and lipid bound sialic acid (LBSA) concentrations, expressed per organ, did not change with time or among groups and a high variability is found especially for those gangliosides at low concentrations. However, the total content of LBSA and gangliosides increased over time for all three groups. Therefore, LBSA and gangliosides increased in brain as a function of brain growth and no enrichment of per gram of tissue occurs over time.

### Retina Composition

There are no significant differences in fatty acid composition of retina between the feeding groups. Similar trends to brain are found in retina regarding the time-course of fatty acid percentages except that percent of 22:6n-3 increased overtime. This result is in agreement with the important role of this fatty acid for retina development.

There are no significant differences among groups at any time or among times within each group as to the content of LBSA, total gangliosides, and main gangliosides classes in the retina. The same is true for the total content of phospholipids and main individual classes, phosphatidylcholine (PC) and phosphatidylethanolamine (PE). In spite of the lack of significant differences, it is still notable that these important lipids tended to increase with time and that the higher content is found after a week of feeding in group B.

### Brain Histology

Neuronal migration and development and maturation of the central nervous system are evaluated. The macroscopic and microscopic analysis of the brains showed neither gross lesions (hemorrhages, ischemic areas, malformations or neoplastic lesions) nor signs of disease.

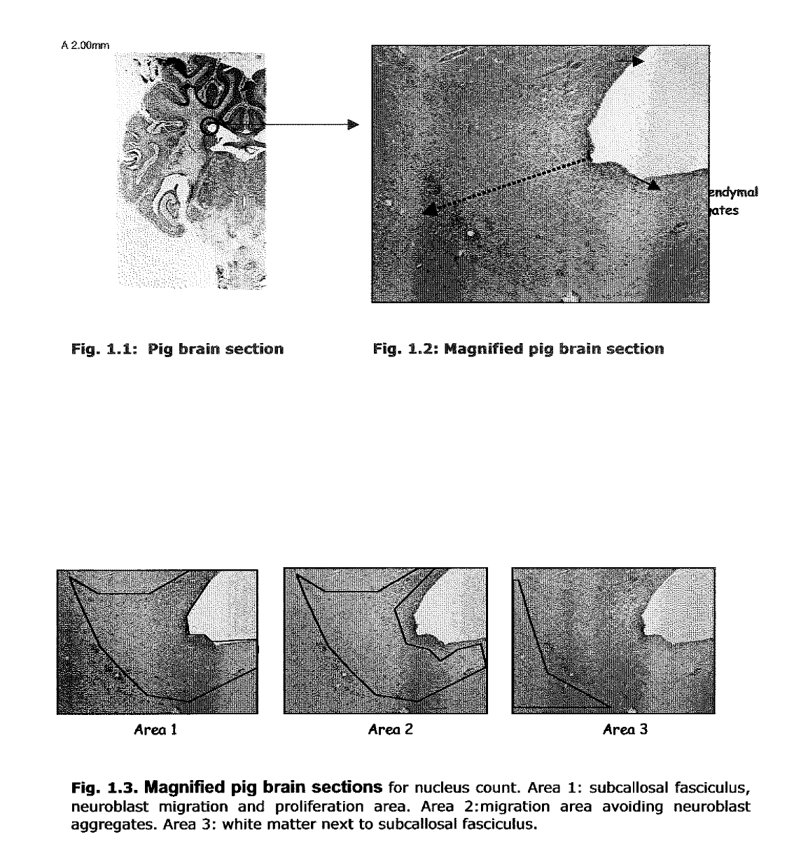

Routine histological techniques are used to quantify the total cell number in selected fields of subcallosal fasciculus and adjacent white matter. This area is selected because neuroblasts migrate and differentiate through several layers just behind the ependymo (see FIGS. 1.1 and 1.2). Nucleus count is done in three different areas of the subcallosal fasciculus (see FIGS. 1.2 and 1.3):
Area 1: migration and proliferation area adjacent to ventriculus lateralis
Area 2: area 1 avoiding neuroblast aggregates in the ependymo (see FIG. 1.3).
Area 3: white matter next to subeallosal fasciculus.

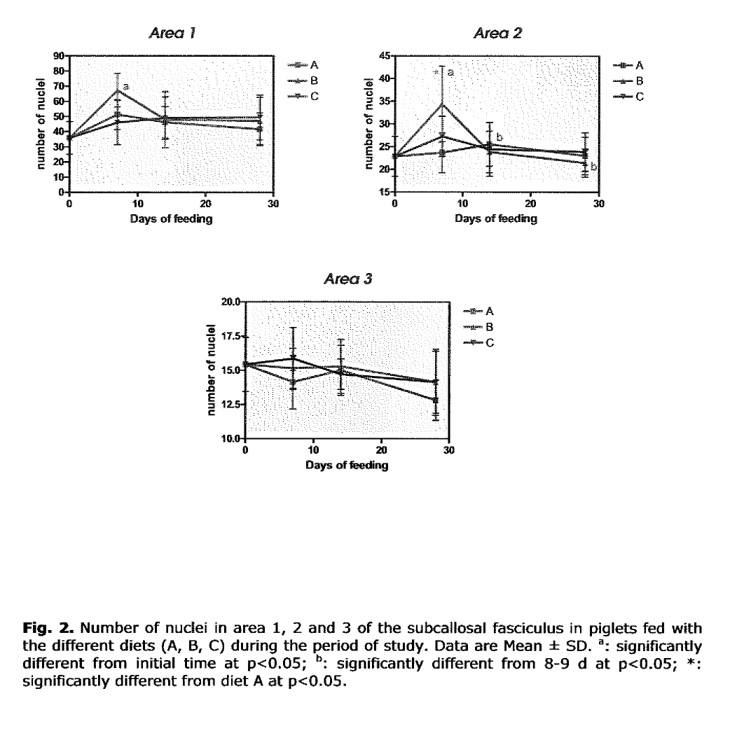

In Area 1, regardless of dietary group, there is a peak in the number of nuclei at 8-9 days of feeding. This peak is mainly due to the higher number of nuclei in the group B at this time (FIG. 2), although differences with other groups did not reach statistical significance (p=0.108 vs. group C). This is likely due to aggregation of stained nuclei next to the border of the lateral ventriculus that increased the variability of the measurement. When the area of aggregated nuclei is avoided (measurement in area 2) the same pattern is obtained, with a reduced variability; thus the number of nuclei in group B is higher than in the other groups being significantly different from group A. No differences are found in area 3.

### Conclusions

There are no significant differences among groups at any time point for contents of protein, DNA and cholesterol in brains. Increases in brain protein and cholesterol contents over time reflect the normal processes of brain growth and myelinization, respectively, that took place during the period of study.

The fatty acid composition of retina followed a similar trend to that found in brain, with no significant differences among groups and similar time-course of fatty acid percentages except for 22:6n-3, which increased overtime. There are no significant differences among groups at any time or among times within each group for the total retina content of lipid-bound sialic acid, gangliosides and phospholipids as well as for individual gangliosides and phospholipids. In spite of the lack of significant differences, it is important to point out that a higher content of all these lipids is found at 8-9 days of feeding for group B. In fact, splitting out the experimental design and performing 1-way ANOVA at 8-9 days among groups A, B, and C, significant differences are found for a higher content of total phospholipids and fatty acids, as well as of phosphatidylethanolamine, and of 20:4n-6 and 22:6n-3 fatty acids in group B.

In the brain histological analysis of total cell number in selected fields of subcallosal fasciculus and adjacent white matter, an area of neuroblast migration, a higher number of nuclei for group B is detected. This transient effect is due to a higher proportion of neuroblast migration at 8-9 days of feeding (12-13 days of life) in animals fed the diet B containing both Lacprodan MFGM-10 and higher levels of arachidonic and docosahexaenoic acids.

Results described in conclusions 2 and 3 above suggest a potential effect of diet B (containing both Lacprodan MFGM-10 and higher levels of arachidonic and docosahexaenoic acids) on neural and visual development. The fact that these effects are not found in group C also containing Lacprodan MFGM-10 or in group A containing the same levels of arachidonic and docosahexaenoic acids, pointed out a synergistic effect of both ingredients (Lacprodan MFGM-10 and arachidonic and docosahexaenoic acids) only when arachidonic and docosahexaenoic acids are at least at the level used in diet B. This suggests a causative role of the diet B ingredients (gangliosides, phospholipids, n-acetylneuraminic acid, and high arachidonic and docosahexaenoic acid concentrations (especially gangliosides and docosahexaenoic acid) in neural migration and neurite growth.

### EXPERIMENT 2

A second animal study is conducted, similar in protocol to that used in Experiment 1, except that this study compares the performance benefits of the following feedings:
Diet A (Group A): Infant formula of the present invention with 0.4% arachidonic and 0.2% docosahexaenoic acid, by weight of total formula fatty acids, and enriched whey protein concentrate at a level of 6.4 g/L of formula on an as-fed basis.
Diet B (Group B): Similac® Advance® Infant Formula, available from Abbott Laboratories, Columbus, Ohio, USA (0.4% arachidonic acid, 0.15% docosahexaenoic acid, by weight of total fatty acids, and conventional whey protein concentrate).
Diet C (Group C): Enfalac® 1 Thailand Infant formula, available from Bristol-Myers Squibb (Thailand) (0-65% arachidonic acid and 0.35% docosahexaenoic acid, by weight of total formula fatty acids, and conventional whey protein concentrate).
Control Group fed Sow's milk

### Summary

The data from the study show significant neural proliferation at 14-16 days of age in neonatal piglets fed sow's milk.

The data also shows that formulas containing low levels of enriched whey protein concentrates (6.4 g/L as fed) are insufficient to duplicate the accelerated neuroblast migration demonstrated in the first study (Experiment 1) using higher levels of enriched whey protein concentrate (7.1 g/L as fed).

### Experimental Design

The study is longitudinal and includes three groups of piglets fed the experimental diets, A, B or C (see Table 4) with two time points of sacrifice after 7-8 days and 14-15 days of feeding. An additional group of piglets fed sows milk is included in the study as a reference. Animals in the sow's milk group are age-matched to coincide with the sacrifice time points of the animals fed the experimental diets. Animals from the sows milk group are sacrificed at the beginning of the study, after 14-16 days old, and after 23-24 days old.

Sixty domestic piglets (3-4-day old) are supplied by a certified farm. Eight piglets from the sow's milk reference group are sacrificed. Forty-eight of the piglets are paired by weight, litter and sex, and are divided into 3 groups (n=16, n=16, and n=16, respectively). Four of the remaining piglets are randomly allocated to the 3 groups (1 to Group A, 1 to Group B, and 2 to Group C).

The piglets are housed in stainless steels wire cages in a conditioned room at 27° C. The animals are fed four times a day with an adapted pig diet, according to their nutritional requirements for a period of three days. Following the three day adaptation period, the piglets are fed four times a day with one of three experimental diets. The time at which the animals are first fed the experimental diet is considered “Time Zero” in the study.

The following tables show the composition of the standard pig diet and of diets A, B and C:

TABLE 4
Experimental Diets

| | Standard | Standard | Diets A | Diets A | | |
|---|---|---|---|---|---|---|
| | pig diet | pig diet per | and B per | and B per | Diet C | Diet C |
| | per 100 g | 100 ml | 100 g | 100 ml | per 100 g | per 100 ml |
| Protein | 25.5 | 4.79 | 10.9 | 14.0 | 12 | 1.5 |
| Fat | 36.3 | 6.82 | 28.9 | 3.71 | 30 | 3.9 |
| Carbohydrates | 31 | 5.83 | 55.3 | 7.1 | 52 | 6.7 |
| Ash | 5.2 | 0.98 | 2.9 | 0.37 | 3.5 | 0.45 |
| Minerals | | | | | | |
| Na (mg) | 201.9 | 37.96 | 126 | 16 | 147 | 19 |
| K (mg) | 800 | 150.40 | 552 | 71 | 620 | 80 |
| Cl (mg) | 300 | 56.40 | 342 | 44 | 390 | 50 |
| Fe (mg) | 32.7 | 6.15 | 9.5 | 1 | 9.4 | 1 |
| Zn (mg) | 13 | 2.44 | 3.94 | 1 | 5.8 | 1 |
| Cu (mg) | 0.8 | 0.15 | 0.473 | 0.061 | .370 | .048 |
| Mg (mg) | 61.4 | 11.54 | 32 | 4 | 47 | 6 |
| Mn (mg) | 0.5 | 0.09 | 0.05 | 0.006 | .076 | .01 |
| Ca (mg) | 1069 | 200.97 | 410 | 53 | 390 | 50 |
| P (mg) | 792 | 148.90 | 221 | 28 | 260 | 33 |
| I (mcg) | 61.7 | 11.60 | 32 | 4 | 79 | 10 |
| Se (mcg) | 20 | 3.76 | 12 | 2 | 17.3 | 2 |
| Vitamins | | | | | | |
| Vitamin A (IU) | 400 | 75.20 | 1577 | 203 | 470 | 60 |
| Vitamin D (IU) | 53 | 9.96 | 315 | 41 | 310 | 40 |
| Vitamin E (IU) | 5 | 0.94 | 16 | 2 | 9.4 | 1 |
| Vitamin K (mcg) | 21.5 | 4.04 | 42 | 5 | 50 | 6 |
| Thiamine (mg) | 0.2 | 0.04 | 0.53 | .07 | 0.39 | .05 |
| Riboflavin (mg) | 0.5 | 0.09 | 0.79 | 0.1 | 0.85 | 0.1 |
| Pyridoxine (mg) | 0.317 | 0.06 | 0.32 | 0.04 | 0.35 | 0.05 |
| Cyanocobalamine | 3.5 | 0.66 | 1.31 | 0.17 | 2.1 | 0.27 |
| (mcg) | | | | | | |
| Pantothenic acid | 2 | 0.38 | 2365 | 304 | 3000 | 386 |
| (mcg) | | | | | | |
| Folic acid (mcg) | 100 | 18.80 | 79 | 10 | 84 | 11 |
| Biotin (mcg) | 26.5 | 4.98 | 23 | 3 | 14.7 | 2 |
| Niacin (mg) | 3 | 0.56 | 5.5 | 1 | 6.3 | 1 |
| Vitamin C (mg) | 71.25 | 13.40 | 47 | 6 | 120 | 15 |
| Others | | | | | | |
| Nucleotides (mg) | — | — | 56 | 7 | 17 | 2 |
| Energy | 552.7 | 103.91 | 525 | 68 | 523 | 67 |

TABLE 5

| | Diet A | Diet B | Diet C |
|---|---|---|---|
| PSNU 2900¹ Protein, g/L as-fed | 6.4 | 0 | 0 |
| Ganglioside mg/L | 17.2 | 4 | 4.6 |
| Sialic acid mg/L | 157 | 139 | 248 |
| Lipid-bound sialic acid | 4.4 | 1.1 | 0.6 |
| (wt % of total sialic acid) | | | |
| Phospholipid mg/L | 440 | 140 | 850 |
| Prebiotic g/L | 0.8 g/L FOS | 0 | 3.6 |
| | | | (GOS + Inulin) |
| Arachidonic acid - wt % of total | 0.4 | 0.4 | 0.65 |
| fatty acids | | | |
| Docosahexaenoic acid - wt % of | 0.2 | 0.15 | 0.35 |
| total fatty acids | | | |

¹Lacprodan MFGM10, enriched whey protein concentrate, Arla Food Ingredients, Denmark

All diets, once prepared, are used immediately or are stored in inert atmosphere cans at 4° C. and used within 24 hours. Diets are in powder form and are reconstituted with water to 12.85% by weight for Diets A, B, and C. The reconstituted liquid diets are poured on the cage feeders, The remaining liquid is removed and measured and the feeders are cleaned prior to subsequent feedings.

For each group, 8 piglets are sacrificed at 7-8 and 14-15 days after the initiation of feeding with control (Diets B and C) or experimental formulas (Diet A).

### Results

### Withdrawals

Four piglets from each group die. Three of the piglets in group A and one of the piglets in group B die during the period of adaptation. One piglet from group B is excluded from the study, because the piglet is very small and did not grow as the rest of the piglets.

Consequently, at 7-8 days, n for group A is 7, n for group B is 8, and n for group C is 8. At 14-15 days, n for group A is 6, n for group B is 4, and n for group C is 6.

### Body Weight and Dietary Intake

The evolution of body weight and dietary intake is very similar for the 3 different dietary groups. There are no differences in body weight evolution among groups for the duration of the experiment When the intake is represented as accumulated dietary intake there are no differences among groups. The evolution of the food efficiency, calculated as grams of body weight/100 kcal of intake, is higher but not significantly different, in group C than in groups A and B, only for the interval of time between 7 and 14 days. A high variability is observed for the interval of time between 0 and 6 days, but there are no differences among groups.

### Brain Histology

Routine histological techniques are used to quantify the total cell number in selected fields of subcallosal fasciculus and adjacent white matter.

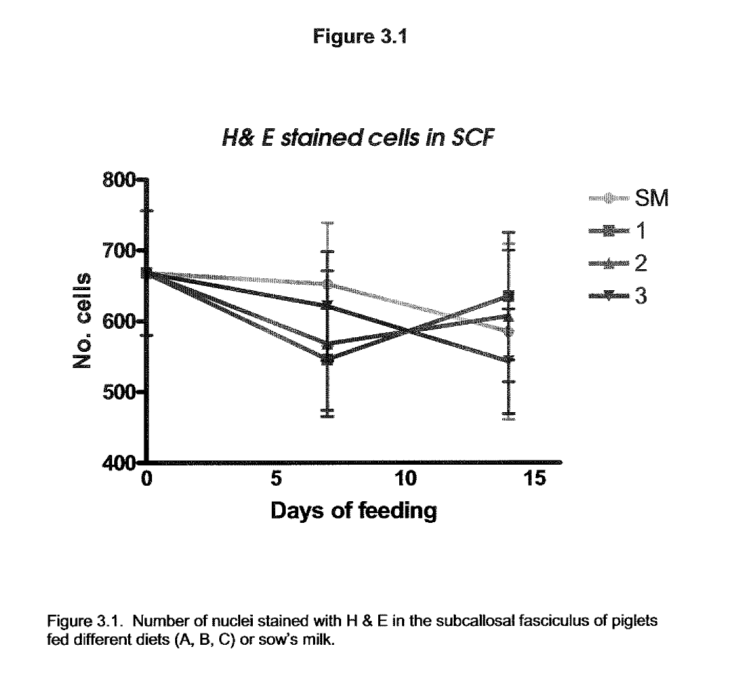

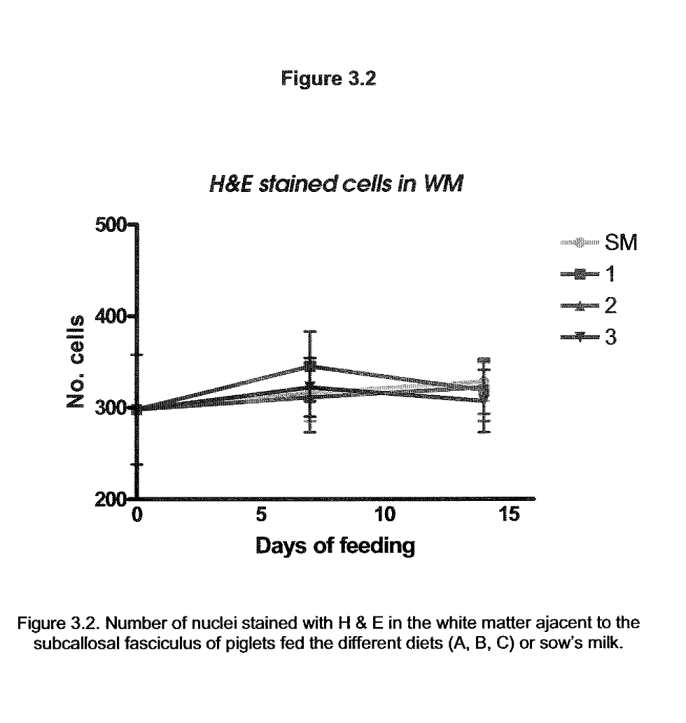

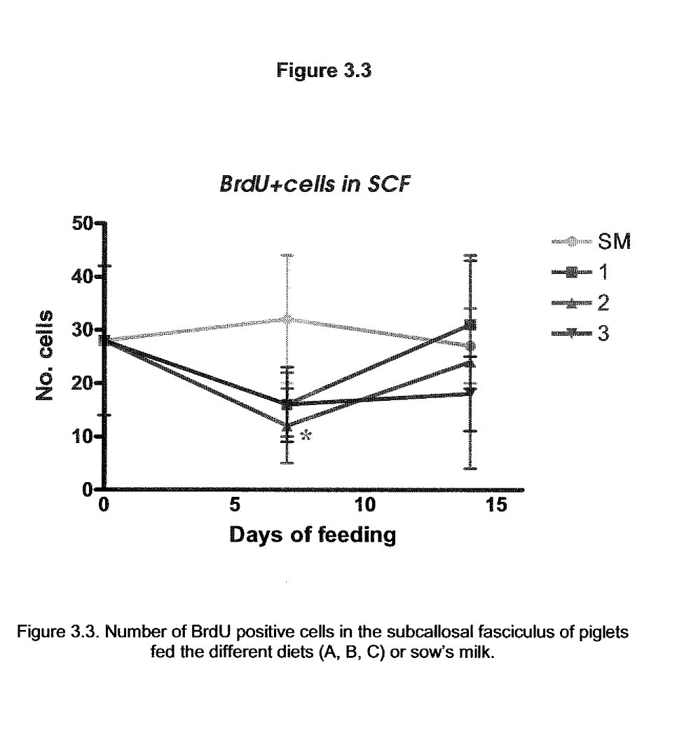

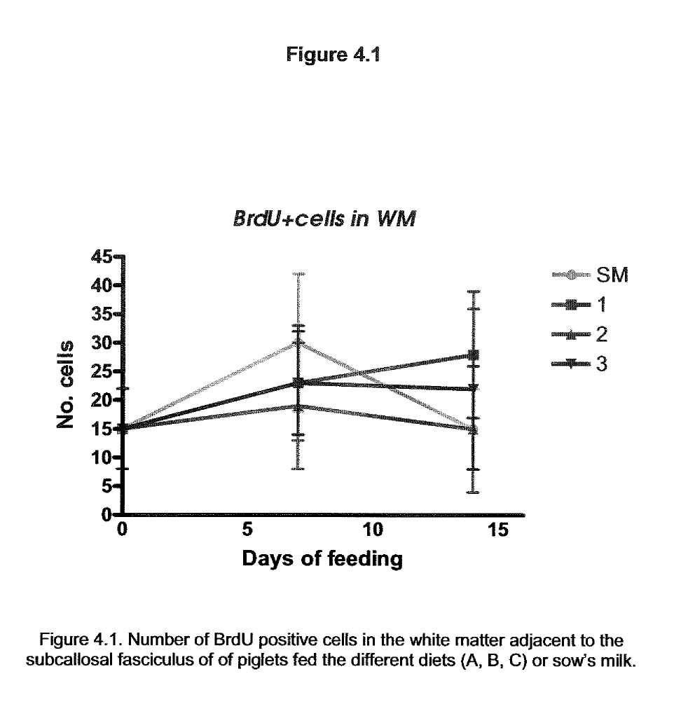

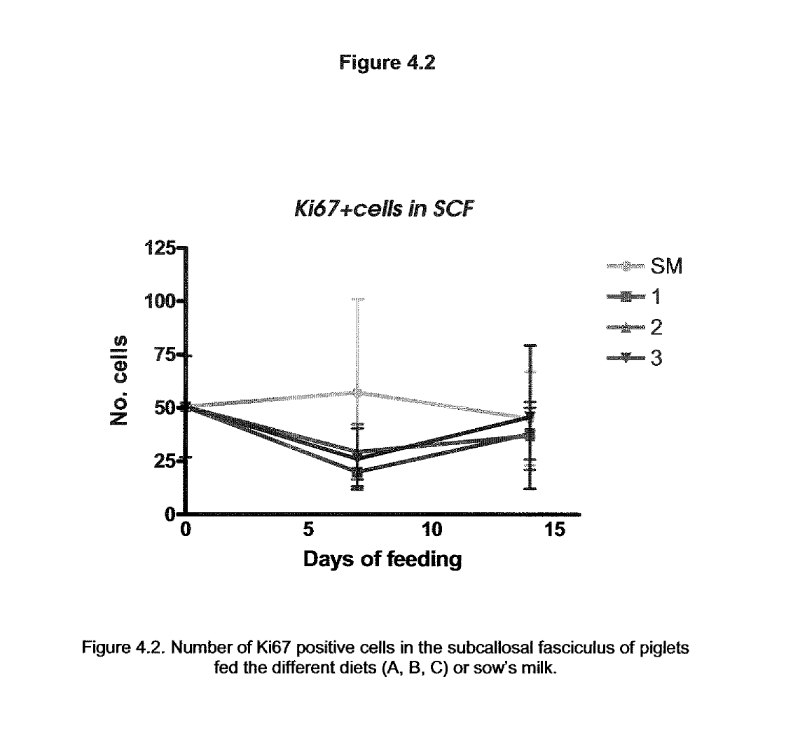

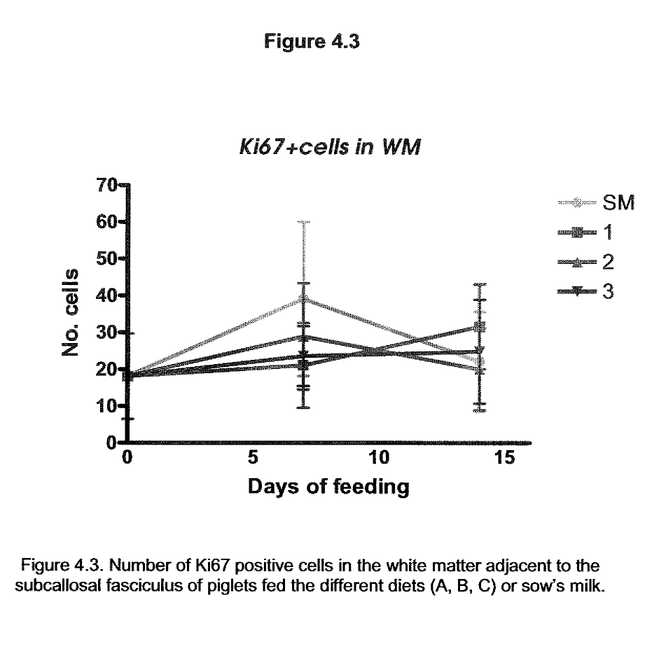

There are no significant differences among groups at any time point in the white matter adjacent to the subcallosal fasciculus (FIG. 3.2, FIG. 4.1, and FIG. 4.3). There are no significant differences among groups at any time point in the number of H&E stained cells in the subcallosal fasciculus (FIG. 3.1). However, there is a higher amount of BrdU and Ki67 stained cells in the subeallosal fasciculus (FIG. 3.3 and FIG. 4.2), at 14-16 days of age, in the sows milk group. The difference between the sow's milk group and Group B is significantly different for the BrdU positive cells.

### Conclusions

Data from Experiments 1 and 2 suggest a synergistic relationship between certain combinations of enriched whey protein concentrate, docosahexaenoic acid, and arachidonic acid, especially based upon the following observations:

Experiment 1 shows that infant formulas (Diet B) with enriched whey protein concentrate (7.1 g/L as fed), docosahexaenoic acid (0.15%) and arachidonic acid (0.4%) accelerate neuroblast migration.

Experiment 1 shows infant formulas (Diet C) with enriched whey protein concentrate (7.1 g/L as fed) and lower concentrations of docosahexaenoic acid (0.1%), and arachidonic acid (0.2%) do not accelerate neuroblast migration.

Experiment 2 shows that infant formulas (Diet A) with docosahexaenoic acid (0.2%) and arachidonic acid (0.4%) and lower levels of enriched whey protein concentrate (6.4 g/L as fed) do not accelerate neuroblast migration.

Experiment 2 also shows that infant formulas (Diet B) with docosahexaenoic acid (0.15%) and arachidonic acid (0.4%), but without enriched whey protein concentrate do not accelerate neuroblast migration.

The results of both experiments therefore show that certain combinations of enriched whey protein concentrate, docosoahexaenoic acid, and arachidonic acid provide accelerated neuroblast migration, provided that their respective concentrations in the formula exceed the threshold levels as defined herein. Conversely, these experiments also show that these components, DHA/ARA and enriched whey protein concentrates, are ineffective for the parameters tested when used alone.

### EXAMPLES

The following examples represent specific embodiments within the scope of the present invention, each of which is given solely for the purpose of illustration and is not to be construed as limitations of the present invention, as many variations thereof are possible without departing from the spirit and scope of the invention. All exemplified amounts are weight percentages based upon the total weight of the composition, unless otherwise specified.

Powder Infant Formulas

The following are powder formula embodiments of the present invention, including methods of using the formula in infants. Ingredients for each formula are listed in the table below.

TABLE 6
Examples 1-4

| | AMOUNT PER 1000 kg OF FORMULA | | | | | | | |
|---|---|---|---|---|---|---|---|---|
| | EXAMPLE 1 | | EXAMPLE 2 | | EXAMPLE 3 | | EXAMPLE 4 | |
| | AA 0.4% | | AA 0.4% | | AA 0.2% | | AA 0.4% | |
| INGREDIENT | DHA 0.2% | | DHA 0.15% | | DHA 0.1% 1 | | DHA 0.2% 1 | |
| LACTOSE | 428.76 | kg | 428.76 | kg | 428.76 | kg | 525.02 | kg |
| NON FAT DRY MILK LOW HEAT | 199.62 | kg | 197.62 | kg | 197.62 | kg | N/A | kg |
| HIGH OLEIC SUNFLOWER OIL | 106.53 | kg | 106.53 | kg | 106.53 | kg | 102.97 | kg |
| COCONUT OIL | 90.74 | kg | 91.09 | kg | 92.87 | kg | 87.57 | kg |
| SOY OIL | 86.37 | kg | 86.37 | kg | 86.37 | kg | 83.49 | kg |
| LACPRODAN MFGM-10 | 51.00 | kg | 53.96 | kg | 53.96 | kg | 154.18 | kg |
| POTASSIUM CITRATE | 7.20 | kg | 7.20 | kg | 7.20 | kg | 7.20 | kg |
| OLIGOFRUCTOSE (FRUCTO- | 7.04 | kg | 7.04 | kg | 7.04 | kg | 7.04 | kg |
| LIGOSACCHARIDE) | | | | | | | | |
| CALCIUM CARBONATE | 4.018 | kg | 4.02 | kg | 4.02 | kg | 9.563 | kg |
| ARACHIDONIC ACID (AA) | 2.87 | kg | 2.87 | kg | 1.44 | kg | 2.87 | kg |
| POTASSIUM CHLORIDE | 1.614 | kg | 1.61 | kg | 1.61 | kg | 1.717 | kg |
| DOCOSAHEXAENOIC ACID (DHA) | 1.40 | kg | 1.05 | kg | 0.70 | kg | 1.40 | kg |
| SODIUM CHLORIDE | 1.303 | kg | 1.30 | kg | 1.30 | kg | 3.280 | kg |
| CHOLINE CHLORIDE | 1.04 | kg | 1.04 | kg | 1.04 | kg | 1.04 | kg |
| ASCORBIC ACID | 766.88 | g | 766.88 | G | 766.88 | g | 766.88 | g |
| VITAMIN PREMIX 25913 | 746.460 | g | 746.46 | G | 746.46 | g | 746.460 | g |
| MAGNESIUM CHLORIDE | 641.63 | g | 641.63 | G | 641.63 | g | 2.18 | g |
| FERROUS SULFATE | 511.98 | g | 511.98 | G | 511.98 | g | 508.79 | g |
| TAURINE | 373.84 | g | 373.84 | G | 373.64 | g | 373.84 | g |
| ASCORBYL PALMITATE | 349.22 | g | 349.22 | G | 349.22 | g | 349.22 | g |
| VITAMIN A, D, RRR-E, K PREMIX | 345.00 | g | 345.00 | G | 345.00 | g | 345.00 | g |
| M-INOSITOL | 254.64 | g | 254.64 | G | 254.64 | g | 254.64 | g |
| CYTIDINE 5′-MONOPHOSPHATE | 243.188 | g | 243.19 | G | 243.19 | g | 243.188 | g |
| DISODIUM URIDINE 5′-MONOP.25% | 192.286 | g | 192.29 | g | 192.29 | g | 192.286 | g |
| DISODIUM GUANOSINE 5′- | 175.452 | g | 175.45 | g | 175.45 | g | 175.452 | g |
| MONOPHO. | | | | | | | | |
| TOCOPHEROL-2 FOOD GRADE | | | | | | | | |
| ANTIOXIDANT | 166.37 | g | 166.37 | g | 166.37 | g | 166.37 | g |
| ZINC SULFATE | 165.70 | g | 165.70 | g | 165.70 | g | 206.02 | g |
| ADENOSINE 5′-MONOPHOSPHATE | 92.043 | g | 92.04 | g | 92.04 | g | 92.043 | g |
| COPPER SULFATE | | | | | | | | |
| ENCAPSULATED | 26.136 | g | 26.14 | g | 26.14 | g | 27.691 | g |
| BETA CAROTENE 30% | 11.64 | g | 11.64 | g | 11.64 | g | 11.64 | g |
| TRICALCIUM PHOSPHATE | 3.000 | g | 3.00 | g | 3.00 | g | 3.000 | g |
| MANGANESE SULFATE | 1.00 | g | 1.00 | g | 1.00 | g | 1.00 | g |
| SODIUM SELENATE | 232.03 | mg | 232.03 | mg | 232.03 | mg | 232.03 | mg |

AA and DHA - percentages by weight of total fatty acids in formula

Each of the exemplified may be prepared in a similar manner by making at least two separate slurries that are later blended together, heat treated, standardized, evaporated, dried and packaged.

Initially, in an oil blend tank, under Nitrogen conditions, an oil slurry is prepared by combining high oleic sunflower oil, soybean oil and coconut oil, followed by the addition of ascorbyl palmitate, beta carotene, vitamin ADEK and mixed tocopherols. The tank is then agitated for 20 minutes and the GA analysis. Following GA clearance and immediately prior to processing the ARA oil, and DHA oil are added to the oil blend tank. The resulting oil slurry is held under moderate agitation at room temperature (<30° C.) for until it is later blended with the other prepared slurry.

Skim milk-oil slurry is prepared by combining the oil blend slurry in approximately 40% of the fluid skim milk at 35-45° C. in a continuous agitation process followed by the addition of an enriched whey protein concentrate. This oil-protein slurry is heated to 65-70° C., two stages homogenised at 154-190/25-45 bars, cooled to 3-6° C. and stored in the process silo.

Skim milk—carbohydrate slurry is prepared by dissolving lactose and Skim milk powder in approximately 60% of the fluid skim milk at 60-75° C. This slurry is held under agitation in the solubilization tank for approximately 2 minutes before pumping to the plate exchanger where is cooled to 3-6° C. and conveyed to the process silo where is blended with the skim milk-oil slurry.

Mineral slurry 1 is prepared by dissolving magnesium chloride, sodium chloride, potassium chloride and potassium citrate in water at room temperature and held under agitation for a minimum of 5 minutes. The mineral slurry 1 is added into the process silo.

Mineral slurry 2 is prepared by dissolving tricalcium phosphate and calcium carbonate in water at 40-60° C. and held under agitation for a minimum of 5 minutes. The mineral slurry 2 added is into the process silo.

Oligofructose slurry is prepared by dissolving oligofructose in water at 40-60° C. and held under agitation for a minimum of 5 minutes. The oligofructose slurry is added into the process silo.

The batch is agitated in the process silo for a minimum of 45 minutes before take a sample for analytical testing. Based on the analytical results of the quality control tests, an appropriate standardization process is carried out.

Vitamin C slurry is prepared by dissolving potassium citrate and ascorbic acid in water at room temperature and held under agitation for a minimum of 5 minutes. The Vitamin C slurry is added into the process silo.

Water-soluble vitamins-inositol slurry is prepared by dissolving potassium citrate, water-soluble vitamin premix and inositol in water at 40-60° C. and held under agitation for a minimum of 5 minutes. The water-soluble vitamin-inositol slurry is added into the process silo.

Ferrous sulphate slurry is prepared by dissolving potassium citrate and ferrous sulphate in water at room temperature and held under agitation for a minimum of 5 minutes.

Nucleotides-choline slurry is prepared by dissolving nucleotide-choline premix in water at room temperature and held under agitation for a minimum of 5 minutes. The nucleotides-choline slurry is added into the process silo.

The final batch is agitated in the process silo for a minimum of 60 minutes before taking a sample for analytical testing. Based on the analytical results of the quality control tests, an appropriate vitamin C and pH correction could be carried out. The final batch is held under moderate agitation at 3-6° C.

After waiting for a period of not longer than 7 days, the resulting blend is preheated to 90-96° C., heated at 110-130° C. for 3 seconds. The heated blend is passed through a flash cooler to reduce the temperature to 93-97° C. and then through an evaporator to achieve the desired solids. The product is then heated to 75-78° C. and pumped to the spray-drying tower. The resulting powder product is collected and stored in bulk powder silos and tested for quality. The finished product is then placed into suitable containers. Samples are taken for microbiological and analytical testing both during in-process and at the finished product stages.

Alternative Process

Each of the exemplified may be prepared in a similar manner by making at least two separate slurries that are later blended together, heat treated, standardized, dried, dry blended and packaged.

Initially, skim milk-mineral slurry is prepared by dissolving approximately 80% of the skim milk powder in demineralized water at 60-65° C., followed by the addition of potassium citrate and potassium hydroxide. The pH of the resulting blend is adjusted to 7.7-8.7 with potassium hydroxide or citric acid.

The rest of the skim milk powder and magnesium chloride is added to the previous blend. The pH of the resulting blend is adjusted to 6.7-7.2 with potassium hydroxide or citric acid.

In a separate tank a new slurry is prepared by dissolving choline chloride and inositol in demineralized water at room temperature. The resulting slurry is combined with the skim milk-mineral slurry and is held under moderate agitation at 60-65° C. for no longer than 1 hour until it is later blended with the additional ingredients.

In a separate tank a new slurry is prepared by dissolving Taurine in demineralized water at 70° C. The resulting slurry is combined with the skim milk-mineral slurry and is held under moderate agitation at 60-65° C. for no longer than 1 hour until it is later blended with the additional ingredients.

An enriched whey protein concentrate is added to the skim milk-mineral slurry followed by lactose and oligofructose. The slurry is agitated in the process silo for a minimum of 30 minutes before take a sample for analytical testing. The pH of the resulting blend is adjusted to 6.5-7.1 with potassium hydroxide or citric acid.

In a oil process tank, under Nitrogen condition, an oil slurry is prepared by combining high oleic sunflower oil, soybean oil and coconut oil, followed by the addition of vitamin ADEK Beta carotene, mixed tocopherols, ascorbyl palmitate, ARA oil, and DHA oil. The resulting oil slurry is held under moderate agitation at room temperature for no longer than six hours until it is later blended with the protein-carbohydrate-mineral slurry.

After waiting for a period of not less than 30 minute nor greater than 6 hours, the protein-carbohydrate-mineral slurry is deaerated at 70-80° C. and further heated to 84-86° C. At this point of the process the oil slurry is injected on line at 50-80° C. The final blend is cooled to 68-72° C. and emulsified through a double stage homogeniser at 145-155 bars in the first stage and at 30-40 bars in the second stage. The heated blend is passed through a plate cooler to reduce the temperature to 3-5° C. and is stored in a process silo.

A mineral solution and an ascorbic acid solution are prepared separately by adding the following ingredients to the processed blended. The mineral solution is prepared by adding the following ingredients to sufficient amount of demineralized water with agitation: citric acid, manganese sulphate, sodium selenate and zinc sulphate. The ascorbic acid solution is prepared by adding ascorbic acid to a sufficient amount of demineralized water to dissolve the ingredient. The processed blend is held under moderate agitation at 3-5° C. for no longer than 48 hours Samples are taken for analytical testing.

The cooled blend is then heated at 69-73° C. and homogenised at 60-70/30-40 bars and sent to the spray drying tower. The base powder product is collected and stored into bulk powder containers. Samples are taken for microbiological and analytical testing.

After the corresponding analytical and microbiological tests are completed, the base powder product is released for the dry blending of the rest of ingredients. The quantities of the remaining ingredients required to obtain the final powder product are determined and entered in the automatic weight system. The system weighs every component of the dry blending premix (Lactose, calcium carbonate, potassium chloride, sodium chloride, water soluble premix, nucleotide cytidine 5-monophosphate, nucleotide disodium uridine 5-monophosphate, nucleotide disodium guanosine 5-monophosphate, nucleotide adenosine 5-monophosphate, copper sulphate and calcium phosphate tribasic. The base powder product and the dry blending premix are conveyed to the blender. The blend is held under agitation for a period of no lees than 20 minutes.

After the blend is completed, the finished product is conveyed to the packaging machine and placed into suitable containers. Samples are taken for microbiological and analytical testing.

The exemplified formulas (Examples 1-4) are non-limiting examples of powder formula embodiments of the present invention. Each formula is reconstituted with water prior to use to a caloric density ranging from about 19 to about 24 kcal/fl oz, and then fed to an infant as a sole source of nutrition during the first 4 months of life, including the first 2 months of life. The formulas help accelerate neural migration, brain development, and cognitive development in the infants.

Liquid Infant Formulas

Examples 1-4 are modified by Conventional means to form ready-to-feed liquid formula embodiments (Examples 5-8) of the present invention The ingredients for Examples 5-8 correspond to the ingredient listings recited in Examples 1-4, respectively.

The exemplified formulas (Examples 5-8) are non-limiting examples of liquid formula embodiments of the present invention. Each formula is adjusted to a caloric density ranging from about 19 to about 24 kcal/fl oz. The finished formula is fed to an infant as a sole source of nutrition during the first 4 months of life, including the first 2 months of life. The formulas help accelerate neural migration, brain development, and cognitive development in the infants.

What is claimed is:

1. Infant formula comprising
(A) at least about 6.5 g/L, on an as-fed basis, of an enriched whey protein concentrate,
(B) at least about 0.13% docosahexaenoic acid by weight of total fatty acids, and
(C) at least about 0.25% arachidonic acid by weight of the total fatty acids.

2. The infant formula of claim 1 wherein the formula includes at least about 5 mg/L of glangliosides, at least about 150 mg/L of phospholipids, and at least about 70 mg/L of total sialic acid with at least about 2.5% by weight of the sialic acid as lipid-bound sialic acid.

3. An infant formula according to claim 2 wherein from about 50% to 100% by weight of the combination of gangliosides, phospholipids, and sialic acid is from an enriched whey protein concentrate.

4. An infant formula according to claim 2 wherein the lipid-bound sialic acid represents from about 2.7% to about 5% by weight of the total sialic acid.

5. An infant formula according to claim 2 comprising, on an as-fed basis, (A) from about 7 mg/L to about 50 mg/L of gangliosides, (B) from about 200 mg/L to about 600 mg/l of phospholipids, and (C) from about 90 mg/l to about 250 mg/L of sialic acid.

6. An infant formula according to claim 1 comprising, by weight of total fatty acids, from about 0.4% to about 2.0% arachidonic acid and from about 0.15% to about 1.0% of docosahexaenoic acid.

7. An infant formula according to claim 2 wherein the total phospholipid comprises at least 20% by weight of sphingomyelin.

8. An infant formula according to claim 7 wherein the phospholipid comprises sphingomyelin, phosphatidyl ethanolamine, phosphatidyl choline, phosphatidyl inositol, and phosphatidyl serine.

9. An infant formula according to claim 2 wherein the formula comprises less than about 0.5% by weight of free glycomacropeptides, on an as-fed basis.

10. An infant formula according to claim 2 wherein the infant formula is substantially free of soy phospholipids, egg phospholipids, and combinations thereof.

11. An infant formula according to claim 2 wherein the formula contains less than about 0.2% by weight of milk fat.

12. An infant formula according to claim 1 wherein the infant formula is a powder.

13. An infant formula according to claim 1 wherein the infant formula is a ready-to-feed liquid.

14. An infant formula according to claim 2 comprising, on an as-fed basis, at least about 190 mg/L of total sialic acid with at least about 2.5% by weight of the sialic acid as lipid-bound sialic acid.

15. A method of accelerating brain development in an infant, comprising
(A) preparing an infant formula comprising
(i) at least about 6.5 g/L, on an as-fed basis, of an enriched whey protein concentrate,
(ii) at least about 0.13% docosahexaenoic acid by weight of total fatty acids, and
(iii) at least about 0.25% arachidonic acid by weight of the total fatty acids, and then
(B) administering or instructing a caregiver to administer the formula to an infant during the first 2 months of life.

16. A method according to claim 15 wherein the infant formula includes, on an as-fed basis:
(A) at least about 5 mg/L of gangliosides,
(B) at least about 150 mg/L of phospholipids, and
(C) at least about 70 mg/L of total sialic acid with at least about 2.5% by weight of the sialic acid as lipid-bound sialic acid.

17. A method according to claim 15 wherein the formula is administered during the first 4 months of life.

18. A method according to claim 16 wherein from about 50% to 100% by weight of the combination of gangliosides, phospholipids, and sialic acid is from the enriched whey protein concentrate.

19. A method according to claim 16 wherein the lipid-bound sialic acid represents from about 2.7% to about 5% by weight of the total sialic acid.

20. A method according to claim 16, wherein the infant formula includes, on an as-fed basis, (A) from about 7 mg/L to about 50 mg/L of gangliosides, (B) from about 200 mg/L to about 600 mg/L of phospholipids, and (C) from about 90 mg/L to about 250 mg/L of sialic acid.

21. A method according to claim 15 wherein the formula comprises, by weight of total fatty acids, from about 0.4% to about 2.0% arachidonic acid and from about 0.15% to about 1.0% of docosahexaenoic acid.

22. A method according to claim 16 wherein the total phospholipid comprises at least 20% by weight of sphingomyelin.

23. A method according to claim 16 wherein the phospholipid comprises sphingomyelin, phosphatidyl ethanolamine, phosphatidyl choline, phosphatidyl inositol, and phosphatidyl serine.

24. A method according to claim 16 wherein the formula contains less than about 0.2% by weight of milk fat.

25. A method according to claim 16 wherein the formula contains less than about 0.5% by weight of free glycomacropeptides.

26. A method according to claim 16 wherein the infant formula is substantially free of soy phospholipids and egg phospholipids.

27. A method according to claim 16 wherein the infant formula includes, on an as-fed basis, at least about 190 mg/L of total sialic acid with at least about 2.5% by weight of the sialic acid as lipid-bound sialic acid.

28. A method of accelerating neural migration in an infant, comprising administering or instructing a caregiver to administer as a sole source of nutrition the infant formula of claim 1 to an infant during the first 4 months of life

29. A method of accelerating vision development in an infant, comprising administering or instructing a caregiver to administer as a sole source of nutrition the infant formula of claim 1 to an infant during the first 4 months of life.

30. A method of accelerating cognitive development in an infant, comprising administering or instructing a caregiver to administer as a sole source of nutrition the infant formula of claim 1 to an infant during the first 4 months of life.
